(19) United States
(12) Patent Application Publication
(10) Pub. No.: US 2009/0253733 A1
(43) Pub. Date: Oct. 8, 2009

(54) RAPAMYCIN CARBONATE ESTERS

(75) Inventors: Alan Rhodes, Reading (GB); Shivpal S. Sandhu, Buckinghamshire (GB); Simon J. Onis, Reading (GB); John E. Mckendrick, Reading (GB)

Correspondence Address: DARDI & ASSOCIATES, PLLC, 220 S. 6TH ST., SUITE 2000, U.S. BANK PLAZA, MINNEAPOLIS, MN, 55402, US

(73) Assignee: BIOINTERACTIONS, LTD., Reading (GB)

(21) Appl. No.: 12/061,416

(22) Filed: Apr. 2, 2008

(51) Int. Cl. A61K 31/439; C07D 498/18; C12P 17/18

(52) U.S. Cl. 514/291; 540/456; 435/119

(57) ABSTRACT

Certain embodiments include carbonate esters of rapamycin at position 42 that are synthesized by a lipase catalyzed regio-specific process. These compounds or a pharmaceutically acceptable salt thereof are useful in the treatment of organ and tissue transplant rejection, autoimmune disease, proliferative disorder, restenosis, cancer, or microbial infection.

### FIELD OF THE INVENTION

The inventions are, in general, related to the field of pharmaceuticals, and certain embodiments relate to synthesis and treatment of diseases using the same.

### BACKGROUND

Rapamycin is a macrolide antibiotic (“-mycin”) first discovered as a product of the bacterium Streptomyces hygroscopicus in a soil sample from an island called Rapa Nui, better known as Easter Island. It was originally developed as an antifungal agent. However, it was soon discovered that rapamycin had potent immunosuppressive and antiproliferative properties. Rapamycin was then developed into a relatively new immunosuppressant drug used to prevent rejection in organ transplantation, and is especially useful in kidney transplants. It is marketed under the trade name RAPAMUNE by Wyeth.

Despite its similar name, rapamycin is not a calcineurin inhibitor like tacrolimus or cyclosporin. However, it has a similar suppressive effect on the immune system. Rapamycin inhibits the response to interleukin-2 (IL-2) and thereby blocks activation of T- and B-cells. In contrast, tacrolimus and cyclosporine inhibit the production of IL-2.

The mode of action of rapamycin is to bind the cytosolic protein FK-binding protein 12 (FKBP12) in a manner similar to tacrolimus. However, unlike the tacrolimus-FKBP12 complex which inhibits calcineurin (PP2B), the rapamycin-FKBP12 complex inhibits the mammalian target of rapamycin (mTOR) pathway through direct binding to the mTOR Complex1 (mTORC1). mTOR is also called FRAP (FKBP-rapamycin associated protein) or RAFT (rapamycin and FKBP target). FRAP and RAFT are in fact more accurate names since they reflect the fact that rapamycin must bind FKBP12 first, and only the FKBP12-rapamycin complex can bind FRAP/RAFT/mTOR.

The chief advantage rapamycin has over calcineurin inhibitors is that it is not toxic to kidneys. Transplant patients maintained on calcineurin inhibitors long-term tend to develop impaired kidney function or even chronic renal failure, and this can be prevented by the use of rapamycin instead. It is particularly advantageous in patients with kidney transplants for hemolytic-uremic syndrome as this disease is likely to recur in the transplanted kidney if a calcineurin-inhibitor is used.

Rapamycin can also be used alone or in conjunction with calcineurin inhibitors and/or mycophenolate mofetil, to provide steroid-free immunosuppression regimes. As impaired wound healing is a possible side effect of rapamycin, some transplant centers prefer not to use it immediately after the transplant operation, and start to give it after a period of weeks or months. Its optimal role in immunosuppression has not yet been determined and is the subject of a number of ongoing clinical trials.

### SUMMARY OF THE INVENTION

Compounds comprising the following structure (I), (II), or (III) can be synthesized using lipase as the catalyzing reagent.

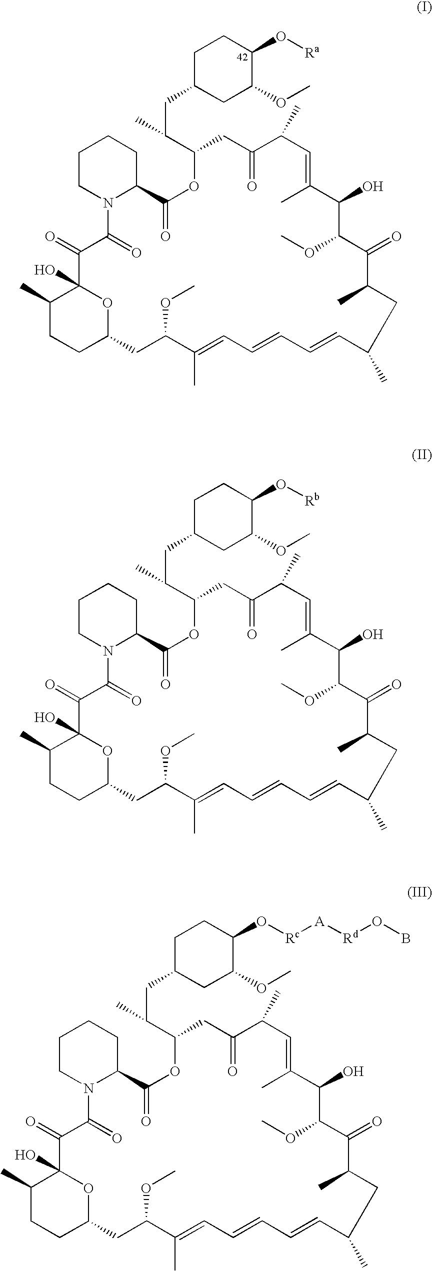

In structures I, II and III,

R^{a} is —C(═O)OR¹, —C(═S)OR¹, —C(═O)SR¹, or —C(═S)SR¹, and,

R¹ is alkyl, alkenyl, alkynyl, cycloalkyl, heteroalkyl, heteroalkenyl, heteroalkynyl, heterocyclyl, or Ar—(CH₂)ₙ— where n is 0 to 12 and Ar is phenyl, substituted phenyl, aryl, heteroaryl, or substituted aryl, where at least one hydrogen of R¹ is replaced by a hydroxyl group, an amino group, a thio group, a phosphate group, a carbonyl group, a sulfonate group, a sulfonamide group, a sulfamide group, a carbonate group or a combination thereof,

R^{b} is —C(═O)OR², —C(═S)OR², —C(═O)SR², or —C(═S)SR² and,

R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group,

R^{c} and R^{d} are each independently —C(═O)O—, —C(═S)O—, —C(═O)S—, or —C(═S)S—,

A is alkyl, alkenyl, alkynyl, cycloalkyl, heteroalkyl, heteroalkenyl, heteroalkynyl, heterocyclyl, and —(CH₂)ₙ—Ar—(CH₂)ₙ— where n is 0 to 12 and Ar is phenyl, substituted phenyl, aryl, heteroaryl, or substituted aryl and,

B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl.

Structures I, II and III also represent pharmaceutically acceptable salts of the compounds. The compounds are expected to be utilized in the treatment of organ and tissue transplant rejection, autoimmune disease, proliferative disorder, restenosis, cancer, or microbial infection.

Structures I and II can be synthesized by reacting a donor with an unprotected hydroxyl group at the '42 position of rapamycin or derivative thereof, using lipase as the catalyst. The donor can be a carbonate, thiocarbonate, or dithiocarbonate. For donors that have reactive side chain such as hydroxyl, amino, thio, phosphate, carbonyl, sulfonate, sulfonamide, sulfamide, or carbonate, the reactive side chain is protected with a protecting group that is removed after the lipase catalyzed reaction. The donor is symmetric, asymmetric or cyclic carbonates including alkyl carbonates, dialkyl carbonates, vinyl carbonates, divinyl carbonates, alkyl vinyl carbonates and cyclic carbonates. In one embodiment, the donor is diethyl carbonate, dioctyl carbonate, ethyl octyl carbonate, diallyl carbonate, cis-octadec-9-enyl vinyl carbonate, divinyl carbonate, 1,4-bis(vinylcarbonate)butane, 1,3-dioxan-2-one, 3-(trimethylsilyloxy)propyl vinyl carbonate, 3-(tert-butyldimethylsilyloxy)propyl vinyl carbonate, (S)-2,2-dimethyl-1,3-dioxolan-4-methyl vinyl carbonate and (S)-2,2-dimethyl-1,3-dioxolan-4-methyl ethyl carbonate.

Structure III can be synthesized by first reacting a bifunctional donor with an unprotected hydroxyl group at the '42 position of rapamycin or derivative thereof to form an adduct, using lipase as the catalyst. The bifunctional donor can be a carbonate, thiocarbonate, dithiocarbonate, or a combination thereof. The bifunctional donor is 1,4-bis(vinylcarbonate)butane, 1,3-bis(vinyl carbonate)propane, 1,2-bis(vinylcarbonate)ethane, 1,4-bis(ethylcarbonate)butane, 1,3-bis(ethyl carbonate)propane, 1,2-bis(ethylcarbonate)ethane, 1,4-bis(methyl vinyl carbonate)cyclohexane or 2,5-bis(methyl vinyl carbonate)furan.

The adduct can be further reacted with a compound of formula B—OH, where OH is the unprotected hydroxyl group and B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl. In one embodiment, the hydroxyl group in B—OH is the 42-hydroxyl of rapamycin or derivative thereof. In one embodiment, B—OH is alkyl alcohols, alkenyl alcohols, alkynyl alcohols, aryl alcohols, diols, triols, polyols, cyclic alcohols, threitol, inositol, or polyethers.

In one embodiment, the compound is 42-O-[(4′-vinyl carbonate]but-1′-oxycarbonyl)rapamycin, 42-O-(cis-octadec-9′-enyloxycarbonyl)rapamycin, 42-O—[(S)-2′,3′-dihydroxypropyloxycarbonyl]rapamycin, 42-O-(3′-hydroxypropoxycarbonyl)rapamycin, 42-O-[(4′-dodecyl carbonate)but-1′-oxycarbonyl]rapamycin.

### DETAILED DESCRIPTION

Rapamycin is a molecule comprising a 31-membered ring including a pipecolinyl group and pyranose ring, a conjugated triene system and a tri-carbonyl region. It also has 15 chiral centers, such that the number of possible stereoisomers is very large. Synthesis involving rapamycin therefore presents many challenges to synthetic chemists.

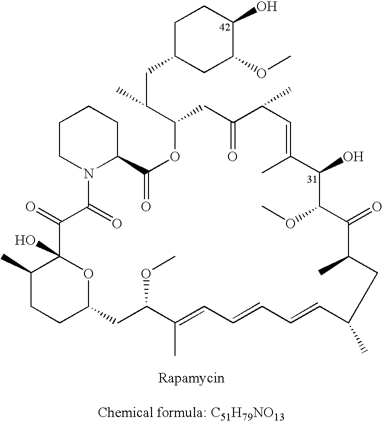

The secondary hydroxyls of rapamycin at positions '31 and '42 respectively are the subject of modifications of appropriate synthetic methodologies. Carbonate esters of rapamycin at position '42 in particular have been shown to have immunosuppressant properties and are useful in the treatment of transplant rejections and autoimmune diseases (U.S. Pat. No. 5,260,300, which discloses certain carbonate esters). Some modifications at the '42 position shows equal or increased potency compared to rapamycin. For example, certain carbonate derivatives at '42 position have demonstrated IC₅₀ equal to or greater than rapamycin in lymphocyte proliferation (LAF) assay.

A number of patents disclose the preparation methods of certain 42-derivatives of rapamycin such as certain alkyl esters (U.S. Pat. No. 4,316,885), certain amino alkyl esters (U.S. Pat. No. 4,650,803), certain fluorinated esters (U.S. Pat. No. 5,100,883), certain amide esters (U.S. Pat. No. 5,118,677), certain carbamate esters (U.S. Pat. No. 5,118,678), certain alkoxy esters (U.S. Pat. No. 5,223,036), certain carbonate esters (U.S. Pat. No. 5,260,300), certain hydroxy esters (U.S. Pat. Nos. 5,362,718 & 6,277,983), and certain rapamycin dimmers afforded through ester linkages (U.S. Pat. No. 5,120,727). However, these preparation methods typically afford poor to moderate yields as a result of poor regio-selectivity and the instability of rapamycin in basic or acidic conditions.

Improvements of the preparation methods have been made. For example, improvement of regio-selectivity through the employment of 31-silyl protected rapamycin was reported (U.S. Pat. No. 6,277,983), which introduced several extra synthetic steps. Additionally, a preparation method through the use of microbial lipases for the catalytic acylation of rapamycin to afford 42-ester derivatives was reported (U.S. Pat. 2005/0234234A1), which made use of acyl donors to afford esters.

Structures I, II and III are carbonate esters of rapamycin, which have been prepared via regio-selective lipase mediated synthesis shown in Scheme I. Carbonate donors were used for the generation of regio-specific carbonate esters to react specifically at the '42 position of rapamycin. For example, mono-hydroxy carbonate esters, polyhydroxy carbonate esters, di-carbonate esters, and carbonate dimers were used as donors to make carbonate esters of rapamycin. The methods disclosed herein are effective at using lipase to add carbonate functionality regio-specifically at 42-position of rapamycin or derivative thereof. The process disclosed herein illustrates improved yields and regio-specificity, for instance, as in comparison with the process disclosed in U.S. Pat. No. 5,260,300.

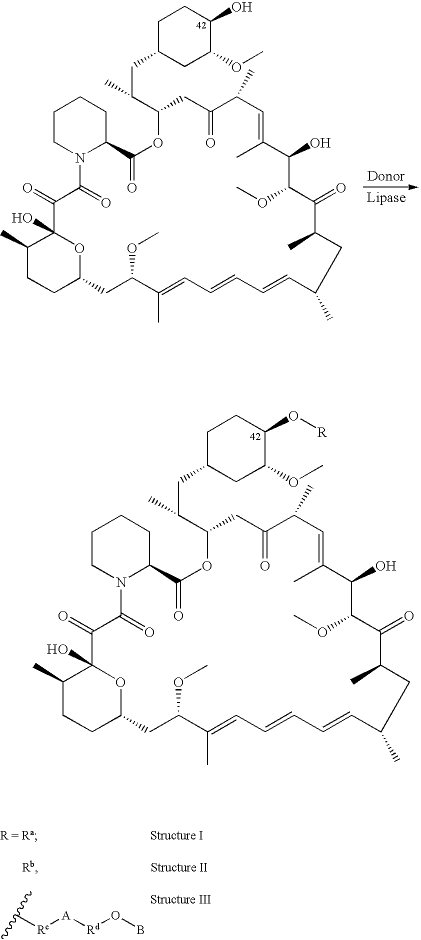

Donors

In one embodiment, the donor is diethyl carbonate, dioctyl carbonate, ethyl octyl carbonate, diallyl carbonate, cis-octadec-9-enyl vinyl carbonate, divinyl carbonate, 1,4-bis(vinylcarbonate)butane, 1,3-dioxan-2-one, 3-(trimethylsilyloxy)propyl vinyl carbonate, 3-(tert-butyldimethylsilyloxy)propyl vinyl carbonate, (S)-2,2-dimethyl-1,3-dioxolan-4-methyl vinyl carbonate and (S)-2,2-dimethyl-1,3-dioxolan-4-methyl ethyl carbonate.

In another embodiment, the donor is bifunctional. The bifunctional donor is 1,4-bis(vinylcarbonate)butane, 1,3-bis(vinylcarbonate)propane, 1,2-bis(vinylcarbonate)ethane, 1,4-bis(ethylcarbonate)butane, 1,3-bis(ethylcarbonate)propane, 1,2-bis(ethylcarbonate)ethane, 1,4-bis(methyl vinyl carbonate)cyclohexane or 2,5-bis(methyl vinyl carbonate)furan.

In one embodiment, the donor is symmetric, asymmetric or cyclic carbonates including alkyl carbonates, dialkyl carbonates, vinyl carbonates, divinyl carbonates, alkyl vinyl carbonates, and cyclic carbonates.

The synthesis discussed in the examples focuses on using carbonate donors. The synthesis involved when other donors such as thiocarbonate, dithiocarbonate are used is known in the art to be similar to the synthesis of carbonate derivatives. This similar synthesis can be adapted by a person of ordinary skill in the art to make other derivatives of rapamycin.

In one embodiment, the donor is a carbonate, O,O′-thiocarbonate, O,S-thiocarbonate, O,S-dithiocarbonate, or S,S′-dithiocarbonate donor of a general structure (IV),

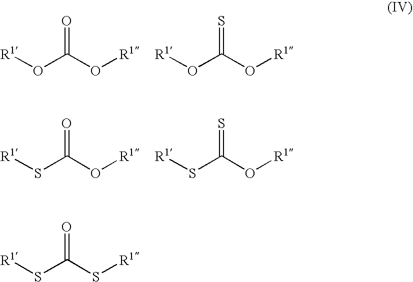

wherein:

R^{1′} is alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl, where at least one hydrogen of R^{1′} is replaced by a hydroxyl group, an amino group, a thio group, a phosphate group, a carbonyl group, a sulfonate group, a sulfonamide group, a sulfamide group, a carbonate group or a combination thereof,

the hydroxyl group, the amino group, the thio group, the phosphate group, the carbonyl group, the sulfonate group, the sulfonamide group, the sulfamide group, or the carbonate group is protected by a protection group, and

R^{1″} is a vinyl, ethyl, propyl, butyl, pentyl, hexyl, heptyl, octyl, or allyl group.

wherein the protection group is removed from the hydroxyl group, the amino group, the thio group, the phosphate group, the carbonyl group, the sulfonate group, the sulfonamide group, the sulfamide group, the carbonate group or a combination thereof after the regio-selective lipase mediated synthesis to make a derivative of rapamycin comprising structure (I).

In one embodiment, the one hydrogen of R^{1′} is replaced by a hydroxyl group. In one embodiment, the R^{1″} is a vinyl group.

In one embodiment, the donor is a carbonate, O,O′-thiocarbonate, O,S-thiocarbonate, O,S-dithiocarbonate, or S,S′-dithiocarbonate donor of a general structure (V),”

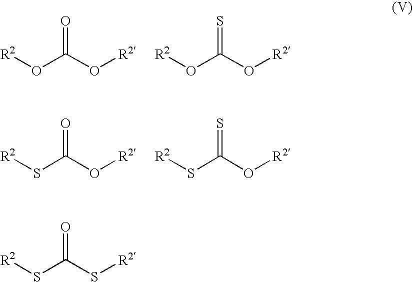

wherein:

R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group,

wherein the aromatic group is Ar—(CH₂)ₙ— where n is 0 to 12 and Ar is phenyl, substituted phenyl, aryl, heteroaryl, or substituted aryl, naphthyl, biphenyl, anthryl, tetrahydronaphthyl, phenanthryl, indane, tetracenyl, pentacenyl, triphenylenyl, imidazolyl, triazolyl, tetrazolyl, oxazolyl, isoxazolyl, thiazolyl, isothiazolyl, thiofuranyl, pyrimidinyl, pyrazinyl, thiazinyl, phenyl substituted with amine, phosphoryl, phosphate, sulfonyl, sulfonate, sulfonamide, and

R^{2′} is a vinyl, ethyl, propyl, butyl, pentyl, hexyl, heptyl, octyl, or allyl group.

The donor of general structure (V) is used in the regio-selective lipase mediated synthesis to make a derivative of rapamycin comprising structure (II). In one embodiment, R^{2′} is a vinyl group.

In one embodiment, the donor is a bifunctional donor of a general structure (VI),

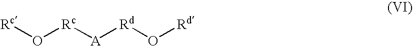

wherein:

R^{c} and R^{d} are each independently —C(═O)O—, —C(═S)O—, —C(═O)S—, or —C(═S)S—,

A is alkyl, alkenyl, alkynyl, cycloalkyl, heteroalkyl, heteroalkenyl, heteroalkynyl, heterocyclyl, and —(CH₂)ₙ—Ar—(CH₂)ₙ— where n is 0 to 12 and Ar is phenyl, substituted phenyl, aryl, heteroaryl, or substituted aryl, and
R^{c′} and R^{d′} are each independently a vinyl, ethyl, propyl, butyl, pentyl, hexyl, heptyl, octyl, or allyl group.

The donor of general structure (VI) is used in the regio-selective lipase mediated synthesis to make a derivative of rapamycin, which is further reacted with a compound of formula B—OH, where OH is a hydroxyl group and B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl, to make a rapamycin derivative comprising structure (III). In one embodiment, the hydroxyl group in formula B—OH is the 42-hydroxyl of rapamycin or derivative thereof. In one embodiment, the B—OH is alkyl alcohols, alkenyl alcohols, alkynyl alcohols, aryl alcohols, diols, triols, polyols, cyclic alcohols, threitol, inositol, or polyethers. In one embodiment, R^{c′} and R^{d′} are each independently a vinyl group.

Chemical name is generally used to describe a substituent, for example alkyl, aryl, etc. Occasionally, the term group is added to the chemical name to describe a substituent, for example carbonyl group, thio group etc. It is understood that both type of descriptions are valid and can be used interchangeably throughout the specification. The term heterocyclic is used herein, meaning a cyclic compound having as a ring member at least two different elements. Cyclic compounds may be aromatic or non-aromatic with at least one ring, e.g., one, two, three, or more rings.

An aromatic group can be any conjugated ring system containing 4n+2 π electrons. There are many criteria available for determining aromaticity. A widely employed criterion for the quantitative assessment of aromaticity is the resonance energy. In some embodiments, the resonance energy of the aromatic group is at least 10 KJ/mol. In further embodiments, the resonance energy of the aromatic group is greater than 0 KJ/mol.

Aromatic groups may be classified as an aromatic heterocyclic group which contains at least a heteroatom in the 4n+2 π-electron ring, or as an arene or aryl group which does not contain a heteroatom in the 4n+2 π-electron ring. Nonetheless, either the aromatic heterocyclic or the arene or aryl group may have at least one heteroatom in a substituent attached to the 4n+2 π-electron ring. Furthermore, either the aromatic heterocyclic or the arene or aryl group may comprise a monocyclic or polycyclic (such as bicyclic, tricyclic, etc.) aromatic ring. An arene is a monocyclic or polycyclic aromatic hydrocarbon; an aryl is formed by removal of a hydrocarbon from a ring carbon atom of an arene.

Non-limiting examples of the aromatic heterocyclic group are furanyl, thiophenyl, pyrrolyl, indolyl, carbazolyl, benzofuranyl, benzothiophenyl, dibenzofuranyl, dibenzothiophenyl, pyridinyl, pyridazinyl, pyrimidinyl, pyrazinyl, triazinyl, tetrazinyl, petazinyl, quinolinyl, isoquinolinyl, cinnolinyl, phthalazinyl, quinazolinyl, quinoxalinyl, naphthyridinyl, pteridinyl, acridinyl, phenanthridinyl, phenanthrolinyl, anthyridinyl, purinyl, pteridinyl, alloxazinyl, phenazinyl, phenothiazinyl, phenoxazinyl, phenoxathiinyl, dibenzo-1,4-dioxinyl, thianthrenyl, and a combination thereof. The aromatic heterocyclic group may also include any combination of the above aromatic heterocyclic groups bonded together either by a bond (as in bicarbazolyl) or by a linking group (as in 1,6 di-10OH-10-phenothiazinyl hexane). The linking group may include an aliphatic group, an aromatic group, a heterocyclic group, or a combination thereof. Furthermore, either an aliphatic group or an aromatic group within a linking group may comprise at least one heteroatom such as O, S, and N. Non-limiting examples of the aryl group are arenaphthyl, biphenyl, anthryl, tetrahydronaphthyl, phenanthryl, indane, tetracenyl, pentacenyl, triphenylenyl, imidazolyl, triazolyl, tetrazolyl, oxazolyl, isoxazolyl, thiazolyl, isothiazolyl, thiofuranyl, pyrimidinyl, pyrazinyl, thiazinyl, phenyl substituted with amine, phosphoryl, phosphate, sulfonyl, sulfonate, or sulfonamide and aromatic heterocyclic group. The aryl group may also include any combination of the above aryl groups bonded together either by a bond (as in biphenyl group) or by a linking group (as in stilbenyl, diphenyl sulfone, an arylamine group). The linking group may include an aliphatic group, an aromatic group, a heterocyclic group, or a combination thereof. Furthermore, either an aliphatic group or an aromatic group within a linking group may comprise at least one heteroatom such as O, S, and N. The term arylamine group includes an N,N-disubstituted arylamine group (e.g., diphenylamine, ethylphenylamine, and diethylamine group), a julolidinyl group, and a carbazolyl group.

An alicyclic compound is a cyclic aliphatic compound having at least one ring, e.g., one, two, three, or more rings. The term aliphatic compound refers to an organic compound that is an alkane or alkene or alkyne or their derivative. Examples of alicyclic compounds include cycloalkanes, e.g., cylcobutane, cyclopentane, cylcohexane, cyclooctane, and bicyclo[2.2.1]heptane group. A heterocyclic non-aromatic compound is a compound having at least one ring and at least two different elements in the ring, e.g., an N, O, or S substituted into at least one ring carbon of cylcohexane, cyclooctane, or bicyclo[2.2.1]heptane group.

The term alkyl, unless otherwise specified, refers to a saturated straight, branched, or cyclic hydrocarbon, and specifically includes, e.g., methyl, ethyl, propyl, isopropyl, butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The alkyl group can be optionally substituted with any appropriate group, including but not limited to one or more groups selected from halo, hydroxyl, amino, alkylamino, arylarmino, alkoxy, aryloxy, nitro, cyano, sulfonic acid, sulfate, phosphonic acid, phosphate, or phosphonate, either unprotected, or protected as necessary, as known to those skilled in the art. The term alkenyl, unless otherwise specified, is a straight, branched, or cyclic (in the case of C₅₋₆) hydrocarbon with at least one double bond, and may be substituted as described above. The term alkynyl, unless otherwise specified, is a hydrocarbon, straight or branched, with at least one triple bond, and may be substituted as described above. In some embodiments, it is useful to limit the size of these substituents to, e.g., less than about 150, less than about 100, less than about 50, or less than about 20 atoms.

Substitution is liberally allowed on the chemical groups, and on the atoms that occupy a position in a formula depicted herein, for various physical effects on the properties of the compounds, such as mobility, sensitivity, solubility, compatibility, stability, and the like, as is known generally in the art. In the description of chemical substituents, there are certain practices common to the art that are reflected in the use of language. The term group indicates that the generically recited chemical entity (e.g., alkyl group, alkenyl group, aromatic group, epoxy group, arylamine group, aromatic heterocyclic group, aryl group, alicyclic group, aliphatic group, heterocyclic non-aromatic group etc.) may have any substituent thereon which is consistent with the bond structure of that group. For example, where the term ‘alkyl group’ is used, that term would not only include unsubstituted linear, branched and cyclic alkyls, such as methyl, ethyl, isopropyl, tert-butyl, cyclohexyl, dodecyl and the like, but also substituents having heteroatom such as 3-ethoxylpropyl, 4-(N-ethylamino)butyl, 3-hydroxypentyl, 2-thiolhexyl, 1,2,3-tribromopropyl, and the like. However, as is consistent with such nomenclature, no substitution would be included within the term that would alter the fundamental bond structure of the underlying group. For example, where a phenyl group is recited, substitution such as 1-aminophenyl, 2,4-dihydroxyphenyl, 1,3,5-trithiophenyl, 1,3,5-trimethoxyphenyl and the like would be acceptable within the terminology, while substitution of 1,1,2,2,3,3-hexamethylphenyl would not be acceptable as that substitution would require the ring bond structure of the phenyl group to be altered to a non-aromatic form because of the substitution. When referring to an epoxy group, the substituent cited includes any substitution that does not destroy the 3-membered ring structure of the epoxy group.

All of these various groups may be optionally derivitized with substituent groups. Suitable substituent groups that may be present on such a “substituted” group include e.g. halogens such as fluoro, chloro, bromo and iodo; cyano; H, hydroxyl group; ester group; ether group; a carbamate, an oxo acid group, an oxo carbon group, an oxo carboxylic acid group, an oxo group, a ketone group; nitro; azido; sulfhydryl; alkanoyl e.g. C₁₋₆ alkanoyl group such as acetyl and the like; carboxamido; alkyl groups, alkenyl and alkynyl groups including groups having one or more unsaturated linkages; alkoxy groups having one or more oxygen linkages; aryloxy such as phenoxy; alkylthio groups; alkylsulfinyl groups; alkylsulfonyl groups; aminoalkyl groups such as groups having one or more N atoms; carbocyclic aryl; aryloxy such as phenoxy; aralkyl having 1 to 3 separate or fused rings; aralkoxy having 1 to 3 separate or fused rings; or a heteroaromatic, heterocyclic, or heteroalicyclic group having 1 to 4 separate or fused rings e.g., with one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl. Other substituents may include groups that include O, S, Se, N, P, Si, C and have between 2 and about 150 atoms. In some embodiments, it is useful to limit the size of any substituent to, e.g., less than about 150, less than about 100, less than about 50, or less than about 20 atoms.

Other suitable substituent groups include these and other N-containing compounds e.g, amines, amides, amidium ions, amine imides, amine oxides, aminium ions, aminonitrenes, nitrenes, aminoxides, nitriles, and nitrile imides. Other suitable substituent groups include these and other S-containing compounds, e.g., sulfonic acid, sulfate, sulfonates, sulfamic acids, sulfanes, sulfatides, sulfenamides, sulfenes, sulfenic acids, sulfenium ions, sulfenyl groups, sulfenylium ions, sulfenyl nitrenes, sulfenyl radicals, sulfides, sulfilimines, sulfimides, sulfimines, sulfinamides, sulfinamidines, sulfines, sulfinic acids, sulfinic anhydrides, sulfinimines, sulfinylamines, sulfolipids, sulfonamides, sulfonamidines, sulfonediimines, sulfones, sulfonic acids, sulfonic anhydrides, sulfonamides, sulfonium compounds, sulfonphthaleins, sulfonylamines, sulfoxides, sulfoximides, sulfoximines, sulfur diimides, thiols, thioacetals, thioaldehydes, thioaldehyde S-oxides, thioanhydrides, thiocarboxylic acids, thiocyanates, thioethers, thiohemiacetals, thioketones, thioketone S-oxides, thiolates, and thionylamines. Other suitable substituent groups include these and other O-containing compounds, e.g., having the form ROH (alcohol), RCOOH (carboxylic acids), RCHO (aldehydes), RR′C═O (ketones), ROR′ (ethers), and RCOOR′ (esters), with the R denoting a bond or atomic element. Other suitable substituent groups include these and other P-containing compounds, e.g., phosphanes, phosphanylidenes, phosphatidic acids, phosphazenes, phosphine oxides, phosphines, phosphinic acids, phosphinidenes, phosphinous acids, phosphoglycerides, phospholipids, phosphonic acids, phosphonitriles, phosphonium compounds, phosphonium ylides, phosphono, phosphonous acids, phosphoramides, and phosphoranes. Carbon is useful for making substituents and the number of carbons in a heteroatomic structure may be, e.g., between 1 and n−1 when between 2 and n atoms are used to form a substituent with, e.g., O, P, S, or N. In some embodiments, it is useful to limit the size of these substituents to, e.g., less than about 150, less than about 100, less than about 50, or less than about 20 atoms.

A variety of substituents are contemplated so that some potential combinations of claimed embodiments may be unstable or impractical to make. A person of ordinary skill in the art can select appropriate stable compounds within the disclosed genus of compounds based on the disclosure herein. Therefore, substituents generally are limited to those substituents that result in appropriate valence for the particular substituted element without forming a charged compound or a radical (except for titratable charged groups, stable zwitterionic forms and triplet neutral radicals with formal unpaired spins with full valencies), as can be conventionally determined by a person of ordinary skill in the art.

Lipase

Lipase is a water-soluble enzyme that catalyzes the hydrolysis of ester bonds. Lipases from fungi and bacteria sources are exploited for various synthetic purposes. For example, Lipase from Candida antarctica “B” Lipase (Novozyme 435) produced by submerged fermentation of a genetically modified Aspergillus oryzae microorganism and absorbed on a macroporous acrylic resin is used in the synthesis of esters and amides, and is known to have broad substrate specificity. A review published in Chemical Review (2001, 101, 2097-2124 by Richard A. Gross et al.) reported lipase catalyzed polycarbonate synthesis.

The Applicants have found lipase can be used to catalyze the addition of carbonate functionality regio-specifically at 42-position of rapamycin or derivative thereof. In one embodiment, the lipase is Novozyme 435 (Sigma-Aldrich, St Louis, Mo.), which is immobilized on macroporous acrylic resin. In another embodiment, the lipase is Amano Lipase PS-C II (Sigma-Aldrich, St Louis, Mo.), which is immobilized on ceramic. In a further embodiment, the lipase is Aspergillus niger lipase, Candida antarctica “A” lipase, Candida antarctica “B” lipase, Amano Lipase PS-C II, Candida rugosa lipase, Mucor miehei lipase, Pseudomonas cepacia lipase (lipase PS), Rhizopus delemar lipase, or alike.

The immobilization of the enzyme on solid support provides added advantages for the overall synthesis. The lipase on solid support could be easily removed from the reaction mixture through simple filtration. Comparable reactions can be performed with the enzyme in solution based on the teachings herein. The product can then be further purified using available chromatographic approaches.

In one embodiment, the process of forming compounds comprising structures I, II and III is performed at about 30-90° C. or in further embodiments from about 40-75° C., for example, for about 1-168 hours in tert-butyl methyl ether (TBME), acetonitrile, toluene or alike. A person of ordinary skill in the art will recognize that additional ranges of reaction temperature and duration within these explicit ranges are contemplated and are within the present disclosure.

Application

Rapamycin-related compounds as described herein may be used for an anti-proliferative effect. In some anti-proliferative embodiments, the compounds are used in conjunction with coronary stents to prevent restenosis in coronary arteries following balloon angioplasty. The compounds may be formulated in a polymer coating that affords controlled release through the healing period following coronary intervention. Several large clinical studies have demonstrated lower restenosis rates in patients treated with rapamycin eluting stents when compared to bare metal stents, resulting in fewer repeat procedures.

In some anti-proliferative embodiments, the compounds are used for treating cancer, either separately or as an adjunct with other therapies. For instance, it was recently shown that rapamycin inhibited the progression of dermal Kaposi's sarcoma in patients with renal transplants. Other mTOR inhibitors such as temsirolimus (CCI-779) or everolimus (RAD001) are being tested for use in cancers such as glioblastoma multiforme and mantle cell lymphoma. Further, combination therapy of doxorubicin and rapamycin has been shown to drive AKT-positive lymphomas into remission in mice. Akt signaling promotes cell survival in Akt-positive lymphomas and acts to prevent the cytotoxic effects of chemotherapy drugs like doxorubicin or cyclophosphamide. Rapamycin blocks Akt signaling and the cells lose their resistance to the chemotherapy. Compounds related to rapamycin disclosed herein are accordingly believed to be useful to block Akt signaling.

Other applications for the rapamycin-related compounds are as antimicrobial agents and blockers of cell proliferation, either in vitro or in vivo. Many uses for reagents with these functionalities are known to artisans.

The compounds may be provided as pharmaceutically acceptable salts, or in pharmaceutically acceptable diluents or excipients. Pharmaceutically acceptable salts of the compounds described herein may be synthesized according to methods known to those skilled in this art, see, for example Pharmaceutical Salts: Properties, Selection, and Use, P. Heinrich Stahl (Editor), Camille G. Wermuth (Editor) June 2002. Generally, such salts are prepared by reacting the free base forms of these compounds with a stoichiometric amount of the appropriate acid in water or in an organic solvent, or in a mixture of the two; generally, nonaqueous media like ether, ethyl acetate, ethanol, isopropanol, or acetonitrile are preferred. Lists of some appropriate salts are found, for example, in Remington's Pharmaceutical Sciences, 17th ed., Mack Publishing Company, Easton, Pa., 1985.

In some embodiments, the compounds described herein are used in combination with one or more potentiators and/or chemotherapeutic agents for the treatment of cancer or tumors. Examples and descriptions of potentiators and combination therapies are provided in, for example, U.S. Pat. Nos. 6,290,929 and 6,352,844.

The compounds described herein may be administered as a single active drug or a mixture thereof with other anti-cancer compounds, and other cancer or tumor growth inhibiting compounds. The compounds may be administered in oral dosage forms that include tablets, capsules, pills, powders, granules, elixirs, tinctures, suspensions, syrups, and emulsions. Further, the compounds may be administered in intravenous (bolus or infusion), intraperitoneal, subcutaneous, or intramuscular form.

An effective amount of the compounds described herein are typically to be administered in a mixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The effective amount can be determined as an amount that provides some relief from the symptoms to be alleviated.

Techniques and compositions for making dosage forms useful in the present invention are described, for example, in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). Doses for present compositions will generally be approximately the same as doses used for rapamycin.

Suitable binders, lubricants, disintegrating agents, coloring agents, flavoring agents, flow-inducing agents, and melting agents may be included as carriers, e.g., for pills. For instance, an active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like.

Suitable binders include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, for example, starch, methyl cellulose, agar, bentonite, xanthan gum, and the like.

The compounds may also be used with liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds may also be coupled to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

The active compounds can be administered orally in solid dosage forms, such as capsules, tablets, and powders, or in liquid dosage forms, such as elixirs, syrups, and suspensions. The active compounds can also be administered parentally, in sterile liquid dosage forms.

Capsules may contain the active compound and powdered carriers, such as lactose, starch, cellulose derivatives, magnesium stearate, stearic acid, and the like. Similarly, such diluents can be used to make compressed tablets. Both tablets and capsules can be manufactured as immediate release products or as sustained release products to provide for continuous or long-term release of the active compounds. The deliverable form of the compounds can be sugar coated or film coated to mask any unpleasant taste and protect the tablet from the atmosphere, or enteric coated for selective disintegration in the gastrointestinal tract.

For oral administration as a liquid, the drug components may be combined with any oral, non-toxic, pharmaceutically acceptable inert carrier such as ethanol, glycerol, water, and the like. Example liquid forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Liquid dosage forms may contain, for example, suitable solvents, preservatives, emulsifying agents, suspending agents, diluents, sweeteners, thickeners, and melting agents.

Liquid dosage forms for oral administration can contain coloring and flavoring, as needed. In general, water, suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds described herein may also be administered in intranasal form via use of suitable intranasal vehicles, or via transdermal routes, using those forms of transdermal skin patches known to those skilled in these arts. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

The compounds set forth herein may also be used in pharmaceutical kits for the treatment of cancer, or other purposes, which comprise one or more containers containing a pharmaceutical composition comprising a therapeutically effective amount of the compound. An effective amount can be determined as an amount that provides some relief from the symptoms to be alleviated. Such kits may further include, if desired, one or more of various components, such as, for example, containers with the compound, containers with one or more pharmaceutically acceptable carriers, additional containers, and instructions. The instructions may be in printed or electronic form provided, for example, as inserts or labels, indicating quantities of the components to be administered, guidelines for administration, and/or guidelines for mixing the components.

The method of administration of the compounds set forth herein can be any suitable method that is effective in the treatment of the particular cancer or tumor type being treated. Treatment may be oral, rectal, topical, parenteral or intravenous administration or by injection into a tumor or cancer. The method of applying an effective amount also varies depending on the disorder or disease being treated. It is believed that parenteral treatment by intravenous, subcutaneous, or intramuscular application of the compounds set forth herein, formulated with an appropriate carrier, additional cancer inhibiting compound or compounds or diluent to facilitate application will be the preferred method of administering the compounds to mammals.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. All patents, patent applications, and publications referenced herein are hereby incorporated by reference herein to the extent that the incorporated material is not contrary to any of the explicit disclosure herein.

The following examples illustrate synthetic application of the above discussed methodology. Examples 1-7 are directed to the synthesis of particular embodiments of compounds. Example 6 used Amano Lipase PS-C II. Example 7 formed a dicarbonate from a rapamycin carbonate substrate. The synthesis of compounds in examples 8-10 is shown to be superior to other synthesis approaches based on yields and regio selectivity.

### EXAMPLES

The following materials were purchased from Sigma-Aldrich Company, Fischer Scientific, Inc., Hichrom Limited and LC Laboratories and were used as received. Ultra-pure water was used throughout the experimental. Thin layer chromatography was carried out on Fluka silica gel F₂₅₄ aluminium backed plates. The plates were visualised by the quenching of Ultra Violet fluorescence (λₘₐₓ=254 nm) and then permanent staining by a solution of vanillin. Silica gel (particle size 20-50 μm) was used for all column chromatography. ¹H NMR spectra were recorded at 250 MHz or 400 MHz and ¹³C NMR spectra were recorded at 62.5 MHz or 100 MHz using a Bruker DPX250 or AMX400 spectrometer. Deuterated chloroform (CDCl₃) was supplied by Cambridge Isotope Laboratories, Inc. and used as solvent. Chemical shifts (δ values) were reported in parts per million (ppm) and all coupling constants (J) were rounded to the nearest 0.5 Hz. C* denotes a quaternary carbon. Accurate mass data was recorded on either a Finnigan MAT 95 under chemical ionisation (CI) conditions using gaseous ammonia or a Bruker micrOTOF under electrospray ionisation (ESI) conditions.

### Example 1

### 42-O-[(4′-Vinyl carbonate)but-1′-oxycarbonyl]rapamycin

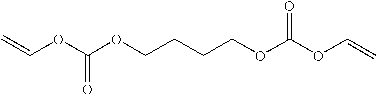

Vinyl chloroformate (2.26 mL, 25 mmol) was slowly added to a stirring solution of 1,4-butanediol (0.98 mL, 11 mmol) in anhydrous pyridine (6.00 mL, 74 mmol) at 0° C. under an atmosphere of N₂ over a period of 30 minutes. The reaction mixture was stirred for a further 1 hour at 0° C. then allowed to warm to room temperature over a period of 1 hour. The temperature was then raised to 50° C. and stirring was continued for a further 1 hour. The reaction was quenched with 14% HCl (40 mL) and the aqueous layer extracted with CH₂Cl₂ (3×40 mL). The combined organic layers were washed with H₂O (2×50 mL) and dried (MgSO₄). The solvent was removed in vacuo and the compound purified by column chromatography (hexane/ether, 1:1) to afford 1,4-bis(vinylcarbonate)butane as a colourless oil (94%, 2.38 g). R_{f}=0.56 (hexane/ether, 1:1). ¹H NMR (250 MHz/CDCl₃); 1.80-1.85 (m, 4H, OCH₂CH₂CH₂CH₂O), 4.24 (t, 4H, OCH₂CH₂CH₂CH₂O, J=7.5 Hz), 4.59 (dd, 2H, 2×OCH═CH trans, J=6.0, 2.0 Hz), 4.92 (dd, 2H, 2×OCH═CH cis, J=14.0, 2.0 Hz), 7.08 (dd, 2H, 2×OCH═CH₂, J=14.0, 6.0 Hz). ¹³C NMR (60 MHz/CDCl₃); 25.4 (OCH₂CH₂CH₂CH₂O), 68.2 (OCH₂CH₂CH₂CH₂O), 98.3 (2×OCH═CH₂), 143.0 (2×OCH═CH₂), 153.1 (2×C═O). HRMS m/z (CI, NH₃) found 231.0879 [M+H]⁺, requires C₁₀H₁₅O₆ 231.0869.

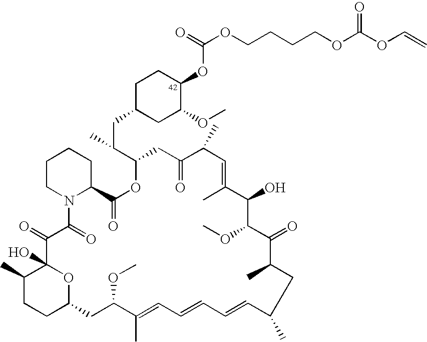

A mixture of rapamycin (0.15 g, 0.16 mmol), 1,4-bis(vinylcarbonate)butane (0.23 g, 0.98 mmol) and Novozyme 435 (0.15 g) in anhydrous tert-butyl methyl ether (TBME) (2.5 mL) was stirred at 60° C. under an atmosphere of N₂ for 18 hours. The enzyme was filtered off, washed with TBME and the combined organic solvent concentrated under N₂. The residue was purified by column chromatography (hexane/acetone, 3:1) to furnish the title compound as a white solid (86%, 0.155 g). R_{f}=0.32 (hexane/acetone, 2:1). ¹H NMR (400 MHz/CDCl₃); 1.70-1.89 (m, 4H), 4.19 (m, 2H), 4.24 (t, 2H), 4.49-4.56 (m, 1H), 4.59 (dd, 1H), 4.92 (dd, 1H), 7.08 (dd, 1H). ¹³C NMR (100 MHz/CDCl₃); 25.1, 25.1, 67.8, 68.0, 80.1, 97.8, 142.6, 152.7, 155.2. MS (ESI-TOF) m/z 1122.6 [M+Na]⁺.

### Example 2

### 42-O-(cis-Octadec-9′-enyloxycarbonyl)rapamycin

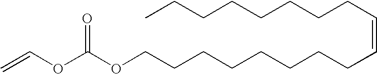

Vinyl chloroformate (0.29 mL, 3.13 mmol) was slowly added to a solution of oleyl alcohol (0.50 mL, 1.58 mmol) in anhydrous pyridine (5 mL, 62 mmol) at 0° C. under an atmosphere of N₂ over a period of 30 minutes. The reaction mixture was stirred for a further 1 hour at 0° C. then allowed to warm to room temperature over a period of 1 hour. The temperature was then raised to 50° C. and stirring was continued for a further 1 hour. The reaction was quenched with 15% HCl (35 mL) and the aqueous layer extracted with CH₂Cl₂ (3×35 mL). The combined organic layers were washed with H₂O (2×50 mL) and dried (MgSO₄). The solvent was removed in vacuo and the compound purified by column chromatography (hexane/ether, 5:1) to afford cis-octadec-9-enyl vinyl carbonate as a colourless liquid (95%, 0.50 g). R_{f}=0.82 (hexane/ether, 3:1). ¹H NMR (250 MHz/CDCl₃); 0.88 (t, 3H, CH₃CH₂CH₂, J=6.5 Hz), 1.27 (brs, 16H, CH₃CH₂CH₂CH₂CH₂CH₂CH₂CH₂CH₂═CHCH₂CH₂CH₂CH₂CH₂CH₂CH₂CH₂O), 1.30 (brs, 6H, CH₂CH₂CH₂CH₂CH₂CH₂CH₂CH═CHCH₂CH₂CH₂CH₂CH₂CH₂CH₂CH₂O), 1.69 (p, 2H, CH₂CH₂O(C═O)OCH═CH₂, J=7.0 Hz), 1.97-2.05 (m, 4H, CH₂CH═CHCH₂), 4.19 (t, 2H, CH₂O(C═O)OCH═CH₂, J=6.5 Hz), 4.57 (dd, 1H, CH₂O(C═O)OCH═CH trans, J=6.0, 2.0 Hz), 4.91 (dd, 1H, CH₂O(C═O)OCH═CH cis, J=14.0, 2.0 Hz), 5.28-5.41 (m, 2H, CH₂CH═CHCH₂), 7.09 (dd, 1H, CH₂O(C═O)OCH═CH₂, J=14.0, 6.0 Hz). ¹³C NMR (60 MHz/CDCl₃); 14.5 (CH₃), 23.1 (CH₂CH₃), 26.0 ((C═O)OCH₂CH₂CH₂), 27.6 (CH₂CH═CHCH₂), 27.6 (CH₂CH═CHCH₂), 28.9 ((C═O)OCH₂CH₂), 29.6 ((C═O)OCH₂CH₂CH_{2CH}₂), 29.7 (CH₂CH₂CH₂CH₃), 29.8 (CH₂CH₂CH₂CH₂CH₃), 29.8 ((C═O)OCH₂CH₂CH₂CH₂CH₂), 29.9 ((C═O)OCH₂CH₂CH₂CH₂CH₂CH₂), 29.9 (CH₂CH₂CH₂CH₂CH₂CH₃), 30.1 ((C═O)OCH₂CH₂CH₂CH₂CH₂CH₂CH₂), 30.2 (CH₂CH₂CH₂CH₂CH₂CH₂CH₃). 32.2 (CH₂CH₂CH₃), 69.2 (CH₂═CHO(C═O)OCH₂), 98.0 (CH₂═CHO), 130.2 (CH₂CH═CHCH₂), 130.4 (CH₂CH═CHCH₂), 143.0 (CH₂═CHO), 153.2 (C═O). HRMS m/z (CI, NH₃) found 339.2902 [M+H]⁺, requires C₂₁H₃₉O₃ 339.2899.

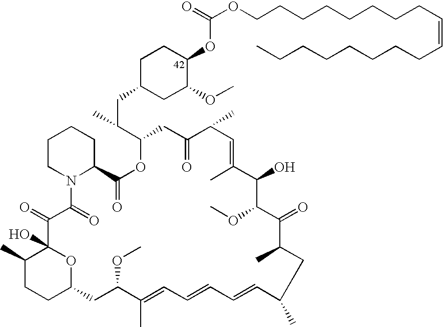

A mixture of rapamycin (0.03 g, 0.0328 mmol), cis-octadec-9-enyl vinyl carbonate (0.067 g, 0.20 mmol) and Novozyme 435 (0.03 g) in anhydrous tert-butyl methyl ether (TBME) (0.5 mL) was stirred at 60° C. under an N₂ atmosphere for 18 hours. The enzyme was filtered off, washed with TBME and the combined organic solvent concentrated under N₂. The residue was purified by column chromatography (hexane/acetone, 4:1) to furnish the title compound as a white solid (78%, 0.031 g). R_{f}=0.71 (THF/heptane, 1:1). ¹H NMR (400 MHz/CDCl₃); 0.88 (t, 3H), 1.26-1.50 (m, 22H), 1.69-1.87 (m, 2H), 1.94-2.03 (m, 4H), 4.13 (t, 2H), 4.49-4.55 (m, 1H), 5.33-5.36 (m, 2H). ¹³C NMR (100 MHz/CDCl₃); 14.1, 22.7, 23.9, 28.6, 28.6, 29.1, 29.2, 29.3, 29.4, 29.4, 29.5, 29.5, 29.8, 31.2, 31.9, 68.1, 80.0, 129.8, 130.0, 155.0. MS (ESI-TOF) m/z 1230.8 [M+Na]⁺.

### Example 3

### 42-O—[(S)-2′,3′-Dihydroxypropyloxycarbonyl]rapamycin

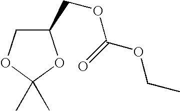

Ethyl chloroformate (1.15 mL, 12 mmol) was slowly added dropwise to a solution of (R)-2,2-dimethyl-1,3-dioxolan-4-methanol (0.8 g, 6.05 mmol) in anhydrous pyridine (6 mL, 74 mmol) at 0° C. under an atmosphere of nitrogen over a period of 30 minutes. The reaction mixture was stirred for a further 1 hour at 0° C., then at room temperature for 1 hour and finally at 50° C. for 1 hour. The mixture was diluted with H₂O (40 mL) and the aqueous layer extracted with CH₂Cl₂ (3×35 mL). The combined organic layers were washed with H₂O (2×50 mL), dried (MgSO₄) and concentrated in vacuo to afford a crude light yellow liquid. The product was purified by column chromatography (hexane/ether, 4:1) to furnish (S)-2,2-dimethyl-1,3-dioxolan-4-methyl ethyl carbonate as a colourless liquid (83%, 1.02 g). R_{f}=0.65 (hexane/ether, 1:1). ¹H NMR (250 MHz/CDCl₃); 1.31 (t, 3H, OCH₂CH₃, J=7.0 Hz), 1.37 (s, 3H, CH₃), 1.44 (s, 3H, CH₃), 3.79 (dd, 1H, C*OCH₂, J=8.5, 6.0 Hz), 4.09 (dd, 1H, C*OCH₂, J=8.5, 6.5 Hz), 4.16 (dd, 1H, CHCH₂OC═O, J=6.0, 5.0 Hz), 4.18 (d, 2H, OCH₂CH₃, J=7.0 Hz), 4.23 (dd, 1H, CHCH₂OC═O, J=7.0, 4.5 Hz), 4.35 (q, 1H, CH, J=6.0 Hz). ¹³C NMR (60 MHz/CDCl₃); 14.6 (OCH₂CH₃), 25.7 (C*CH₃), 27.1 (C*CH₃), 64.6 (CH₂CH₃), 66.7 (C*OCH₂), 68.1 (CHCH₂OC═O), 73.7 (CH), 110.3 (C*), 155.4 (C═O). HRMS m/z (CI, NH₃) found 205.1083 [M+H]⁺, requires C₉H₁₇O₅ 205.1076.

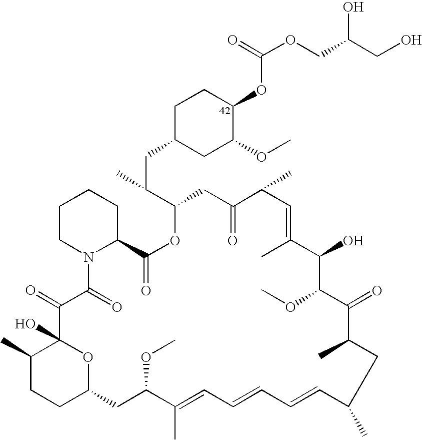

A mixture of rapamycin (0.3 g, 0.328 mmol), (S)-2,2-dimethyl-1,3-dioxolan-4-methyl ethyl carbonate (0.47 g, 2.30 mmol) and Novozyme 435 (0.3 g) in anhydrous tert-butyl methyl ether (TBME) (3.0 mL) was stirred at 60° C. under an N₂ atmosphere for 18 hours. The enzyme was filtered off, washed with TBME and the combined organic solvent concentrated under N₂. The residue was purified by column chromatography (hexane/acetone, 4:1) and then subjected to acid catalysed deprotection. The crude product (0.333 g) was dissolved in THF (3.5 mL), cooled to 0° C. and H₂SO₄ (2.1 mL, 2N) was added dropwise over a period of 25 minutes. The mixture was stirred for 4 hours at 0° C. then at room temperature for a further 20 hours. After TLC had indicated complete consumption of 42-O—[(S)-2′,2′-dimethyl-1′,3′-dioxolan-4′-methoxycarbonyl]rapamycin the mixture was diluted with brine (5 mL) and the aqueous layer was extracted with EtOAc (3×5 mL). The combined organic layers were washed with H₂O (5 mL), 5% NaHCO₃ (5 mL) and then brine (5 mL). The organic layers were dried (MgSO₄) and concentrated in vacuo to afford a crude yellow oil. The product was purified by column chromatography (hexane/acetone, 2:1) to furnish the title compound as a white solid. R_{f}=0.08 (THF/heptane, 1:1). ¹H NMR (400 MHz/CDCl₃); 3.61 (d, 1H), 3.70-3.75 (m, 1H), 3.97 (p, 1H), 4.24 (d, 2H), 4.49-4.56 (m, 1H). ¹³C NMR (100 MHz/CDCl₃); 63.1, 68.5, 69.9, 80.5, 154.9. MS (ESI-TOF) m/z 1054.6 [M+Na]⁺.

### Example 4

### 42-O—[(S)-2′,3′-Dihydroxypropyloxycarbonyl]rapamycin

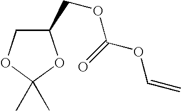

Novozyme 435 (0.1 g) was added to a solution of (R)-2,2-dimethyl-1,3-dioxolan-4-methanol (0.107 g, 0.81 mmol) and divinyl carbonate (0.37 g, 3.22 mmol) in toluene (1.96 mL). The mixture was stirred at 60° C. under N₂ for 16 hours. After TLC had indicated complete consumption of starting material, the enzyme was filtered off and washed with THF. The THF, toluene, acetaldehyde and excess divinyl carbonate were removed in vacuo and the crude product was purified by column chromatography (hexane/ether, 2.5:1) to afford (S)-2,2-dimethyl-1,3-dioxolan-4-methyl vinyl carbonate as a colourless liquid (86%, 0.14 g). R_{f}=0.54 (hexane/ether, 2:1). ¹H NMR (250 MHz/CDCl₃); 1.37 (s, 3H, CH₃), 1.44 (s, 3H, CH₃), 3.81 (dd, 1H, OCH₂, J=5.5, 8.5 Hz), 4.11 (dd, 1H, OCH₂, J=6.5, 8.5 Hz), 4.22 (dd, 1H, (C═O))OCH₂, J=5.0, 7.5 Hz), 4.22 (dd, 1H, (C═O)OCH₂, J=6.0, 8.5 Hz), 4.38 (q, 1H, CH, J=6.0 Hz), 4.60 (dd, 1H, CH═CH₂ trans, J=2.0, 6.0 Hz), 4.93 (dd, 1H, CH═CH₂ cis, J=2.0, 14.0 Hz), 7.08 (dd, 1H, CH═CH₂, J=6.0, 14.0 Hz). ¹³C NMR (60 MHz/CDCl₃); 25.7 (CH₃), 27.0 (CH₃), 66.5 (OCH₂), 68.7 ((C═O)OCH₂), 73.5 (CH), 98.5 (CH═CH₂), 110.4 (C*), 142.9 (CH═CH₂), 153.0 (C═O). HRMS m/z (CI, NH₃) found 203.0920 [M+H]⁺, requires C₉H₁₅O₅ 203.0920.

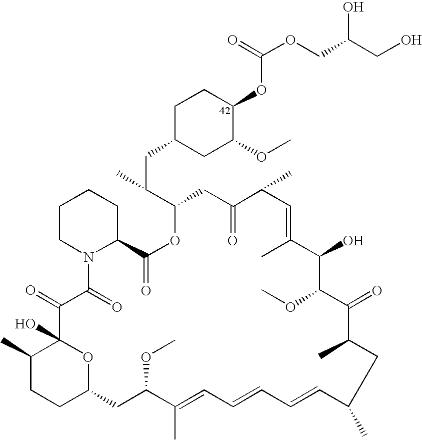

A mixture of rapamycin (0.1 g, 0.109 mmol), (S)-2,2-dimethyl-1,3-dioxolan-4-methyl vinyl carbonate (0.13 g, 0.64 mmol) and Novozyme 435 (0.15 g) in anhydrous tert-butyl methyl ether (TBME) (2.0 mL) was stirred at 60° C. under an N₂ atmosphere for 24 hours. The enzyme was filtered off, washed with TBME and the combined organic solvent concentrated under N₂. The residue was purified by column chromatography (hexane/acetone, 3:1) and then subjected to acid catalysed deprotection. The crude product (0.113 g) was dissolved in THF (1.5 mL), cooled to 0° C. and H₂SO₄ (1.2 mL, 2N) was added dropwise over a period of 25 minutes. The mixture was stirred for 4 hours at 0° C. then at room temperature for a further 20 hours. After TLC had indicated complete consumption of 42-O—[(S)-2′,2′-dimethyl-1′,3′-dioxolan-4′-methoxycarbonyl]rapamycin the mixture was diluted with brine (2 mL) and the aqueous layer was extracted with EtOAc (3×2 mL). The combined organic layers were washed with H₂O (2 mL), 5% NaHCO₃ (2 mL) and then brine (2 mL). The organic layers were dried (MgSO₄) and concentrated in vacuo to afford a crude yellow oil. The product was purified by column chromatography (hexane/acetone, 2:1) to furnish the title compound as a white solid. R_{f}=0.08 (THF/heptane, 1:1). ¹H NMR (400 MHz/CDCl₃); 3.61 (d, 1H), 3.70-3.75 (m, 1H), 3.97 (p, 1H), 4.24 (d, 2H), 4.49-4.56 (m, 1H). ¹³C NMR (100 MHz/CDCl₃); 63.1, 68.5, 69.9, 80.5, 154.9. MS (ESI-TOF) m/z 1054.6 [M+Na]⁺.

### Example 5

### 42-O-(3′-Hydroxypropoxycarbonyl)rapamycin

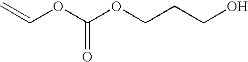

Vinyl chloroformate (0.86 mL, 9.39 mmol) was slowly added dropwise to a solution of 1,3-propanediol (2.14 g, 28 mmol) in anhydrous pyridine (8 mL) at 0° C. over a period of 1 hour. The reaction was stirred for a further 30 minutes at 0° C., then at room temperature for 1 hour and finally at 50° C. for 30 minutes. The reaction mixture was quenched with 14% HCl (40 mL) and the aqueous layer was extracted with CH₂Cl₂ (3×40 mL). The combined organic layers were washed with H₂O (2×60 mL), dried (MgSO₄) and concentrated in vacuo to afford a crude yellow liquid. The product was purified by column chromatography (hexane/ether, 4:1) to afford 3-hydroxypropyl vinyl carbonate as a colourless liquid (73%, 0.997 g). R_{f}=0.36 (hexane/ether, 1:1). ¹H NMR (250 MHz/CDCl₃); 1.97 (p, 2H, CH₂CH₂CH₂, J=5.0 Hz), 2.66 (s, 1H, OH), 3.74 (t, 2H, CH₂OH, J=5.0 Hz), 4.35 (t, 2H, OCH₂CH₂, J=5.0 Hz), 4.59 (dd, 1H, CH═CHO trans, J=7.5, 2.5 Hz), 4.92 (dd, 1H, CH═CHO cis, J=12.5, 2.5 Hz), 7.08 (dd, 1H, CH₂═CH, J=14.0, 5.0 Hz). ¹³C NMR (60 MHz/CDCl₃); 31.4 (CH₂CH₂CH₂), 58.6 (CH₂OH), 65.6 (OCH₂CH₂), 97.9 (C₂═CH), 142.5 (CH₂═CH), 152.9 (C═O). HRMS m/z (CI, NH₃) found 129.0558 [M+H—H₂O]⁺, requires C₆H₉O₃ 129.0552.

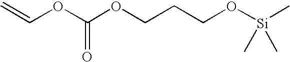

A solution of 3-hydroxypropyl vinyl carbonate (0.456 g, 3.12 mmol) and triethylamine (1.09 mL, 7.8 mmol) in anhydrous EtOAc (12 mL) was cooled to 0° C. and trimethylsilyl chloride (0.79 mL, 6.24 mmol) in anhydrous EtOAc (8 mL) was added over a period of 15 minutes. The reaction was allowed to warm to room temperature and was stirred for an additional 30 minutes. The mixture was then poured onto ice water (30 mL) and the aqueous layer was extracted with EtOAc (3×30 mL). The combined organic layers were washed with H₂O (40 mL), dried (MgSO₄) and concentrated in vacuo to furnish a crude yellow liquid. The product was purified by column chromatography (hexane/ether, 8:1) to afford 3-(trimethylsiloxy)propyl vinyl carbonate as a colourless liquid (78%, 0.53 g). R_{f}=0.73 (hexane/ether, 8:1). ). ¹H NMR (250 MHz/CDCl₃); 0.01 (s, 9H, 3×CH₃), 1.80 (p, 2H, CH₂CH₂CH₂, J=5.0 Hz), 3.58 (t, 2H, CH₂OSi, J=5.0 Hz), 4.20 (t, 2H, OCH₂CH ₂, J=5.0 Hz), 4.46 (dd, 1H, CH═CHO trans, J=7.5, 2.5 Hz), 4.80 (dd, 1H, CH═CHO cis, J=12.5, 2.5 Hz), 6.98 (dd, 1H, CH₂═CH, J=14.0, 5.0 Hz). ¹³C NMR (60 MHz/CDCl₃); 0.0 (3×CH₃), 32.1 (CH₂CH₂CH₂), 59.0 (CH₂OSi), 66.2 (OCH₂CH₂), 98.3 (CH₂═CHO), 143.3 (CH₂═CH), 153.4 (C═O). HRMS m/z (CI, NH₃) found 219.1058 [M+H]⁺, requires C₉H₁₉O₄Si 219.1053.

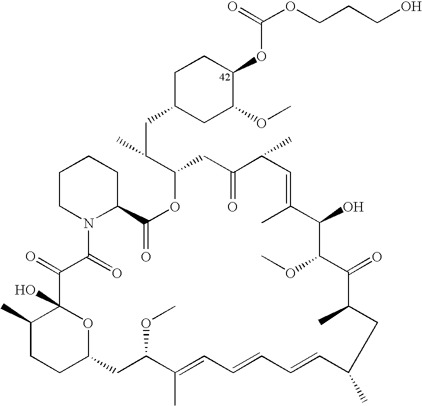

A mixture of rapamycin (0.06 g, 0.066 mmol), 3-(trimethylsiloxy)propyl vinyl carbonate (0.072 g, 0.328 mmol), Novozyme 435 (0.08 g) and molecular sieves (5 Å) in anhydrous tert-butyl methyl ether (TBME) (1.0 mL) was stirred at 60° C. under an N₂ atmosphere for 72 hours. The enzyme was filtered off, washed with TBME and the combined organic solvent concentrated under N₂. The residue was purified by column chromatography (hexane/acetone, 3:1) and then subjected to acid catalysed deprotection. The crude product (0.0649 g) was dissolved in THF (0.6 mL), cooled to 0° C. and H₂SO₄ (0.28 mL, 0.5N) was added dropwise over a period of 15 minutes. Stirring was continued for 2 hours at 0° C. and then at room temperature for a further 1 hour. After TLC had indicated complete consumption of 42-O-[3′-(trimethylsilyloxy)propyloxycarbonyl]rapamycin the mixture was transferred to a separatory funnel and the aqueous layer was extracted with EtOAc (3×2 mL). The combined organic layers were washed with H₂O (2 mL), 5% NaHCO₃ (2 mL) and then brine (2 mL). The organic layers were dried (MgSO₄) and concentrated in vacuo to afford a colourless semi-solid. The product was purified by column chromatography (hexane/acetone, 2:1) to furnish the title compound as a white solid. R_{f}=0.26 (THF/heptane, 1:1). ¹H NMR (400 MHz/CDCl₃); 1.92 (p, 2H), 3.74-3.75 (m, 2H), 4.23-4.27 (m, 2H), 4.49-4.56 (m, 1H). ¹³C NMR (100 MHz/CDCl₃); 31.6, 58.9, 64.3, 80.2, 155.3. MS (ESI-TOF) m/z 1038.5 [M+Na]⁺.

### Example 6

### 42-O-[(4′-Vinyl carbonate)but-1′-oxycarbonyl]rapamycin (Alternative Lipase)

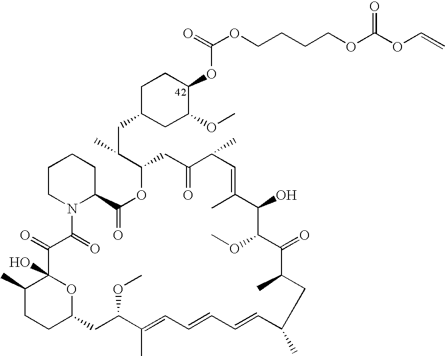

A mixture of rapamycin (0.03 g, 0.0328 mmol), 1,4-bis(vinylcarbonate)butane (0.045 g, 0.197 mmol) and Amano Lipase PS-C II (0.03 g) in anhydrous tert-butyl methyl ether (TBME) (0.5 mL) was stirred at 60° C. under an atmosphere of N₂ for 18 hours. The enzyme was filtered off, washed with TBME and the combined organic solvent concentrated under N₂. The residue was purified by column chromatography (hexane/acetone, 2.5:1) to furnish the title compound as white solid (76%, 0.0276 g). R_{f}=0.32 (hexane/acetone, 2:1). ¹H NMR (400 MHz/CDCl₃); 1.70-1.89 (m, 4H), 4.19 (m, 2H), 4.24 (t, 2H), 4.49-4.56 (m, 1H), 4.59 (dd, 1H), 4.92 (dd, 1H), 7.08 (dd, 1H). ¹³C NMR (100 MHz/CDCl₃); 25.1, 25.1, 67.8, 68.0, 80.1, 97.8, 142.6, 152.7, 155.2. MS (ESI-TOF) m/z 1122.6 [M+Na]⁺.

### Example 7

### 42-O-[(4′-Dodecyl carbonate)but-1′-oxycarbonyl]rapamycin

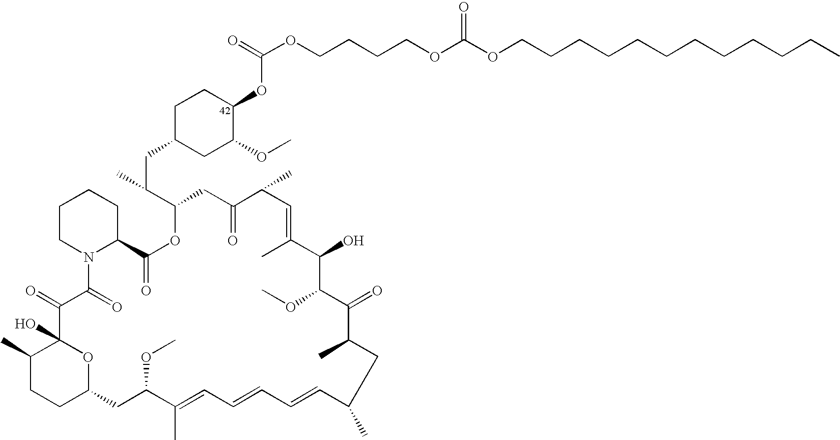

A mixture of 42-O-[(4′-vinyl carbonate]but-1′-oxycarbonyl)rapamycin (0.029 g, 0.0264 mmol), 1-dodecanol (0.025 g, 0.133 mmol), Novozyme 435 (0.045 g) and molecular sieves (5 Å) in anhydrous acetonitrile (0.5 mL) was stirred at 60° C. under an atmosphere of N₂ for 18 hours. The enzyme was filtered off, washed with acetonitrile and the combined organic solvent concentrated under N₂. The residue was purified by column chromatography (hexane/acetone, 4:1) to furnish the title compound as light yellow oil. R_{f}=0.38 (hexane/acetone, 2:1). ¹H NMR (400 MHz/CDCl₃); 0.88 (t, 3H), 1.21-1.39 (m, 14H), 1.29-1.43 (m, 4H), 1.60-1.70 (m, 4H), 1.71-1.81 (m, 2H), 3.62-3.72 (m, 2H), 4.10-4.20 (m, 4H), 449-4.56 (m, 1H). ¹³C NMR (100 MHz/CDCl₃); 14.1, 22.7, 25.2, 25.7, 25.7, 28.7, 28.9, 29.2, 29.3, 29.4, 29.5, 29.6, 31.9, 67.6, 68.2, 70.5, 80.1, 155.3, 155.4. MS (ESI-TOF) m/z 1264.7 [M+Na]⁺.

The following examples are included to illustrate improved yields in comparison with U.S. Pat. No. 5,260,300.

### Example 8

### 42-O-(Ethoxycarbonyl)rapamycin

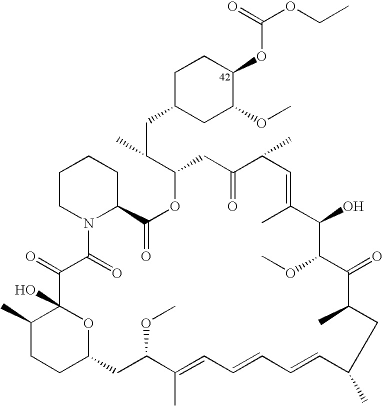

A mixture of rapamycin (0.06 g, 0.0656 mmol), diethyl carbonate (0.047 g, 0.0394 mmol), Novozyme 435 (0.09 g) and molecular sieves (5 Å) in anhydrous tert-butyl methyl ether (TBME) (1.0 mL) was stirred at 60° C. under an N₂ atmosphere for 18 hours. The enzyme was filtered off, washed with TBME and the combined organic solvent concentrated under N₂. The residue was purified by column chromatography (hexane/acetone, 3:1) to furnish the title compound as a white solid (79%, 0.0509 g). R_{f}=0.56 (THF/heptane, 1:1). ¹H NMR (400 MHz/CDCl₃); 1.31 (t, 3H), 4.22 (q, 2H), 4.48-4.55 (m, 1H). ¹³C NMR (100 MHz/CDCl₃); 14.2, 65.8, 80.0, 154.8. MS (ESI-TOF) m/z 1008.6 [M+Na]⁺.

### Example 9

### 42-O-(Vinyloxycarbonyl)rapamycin

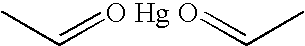

A mixture of mercury(II) oxide yellow (162 g, 750 mmol), mercury(II) acetate (6 g, 25 mmol), ethanol (90 mL) and H₂O (30 mL) was placed in a 500 mL three-necked flask equipped with a magnetic stirrer, thermometer, reflux condenser and addition funnel. The mixture was stirred at room temperature for 30 minutes until homogeneous. Ethyl vinyl ether (118.8 g, 1650 mmol) was added to the reaction mixture over a period of 30 minutes, during which time the temperature rose to 55° C. The reaction mixture was filtered whilst hot then allowed to crystallise for 3 hours at 4° C. The crystals were washed with ethanol and dried under vacuum for 24 hours. Mercuric diacetaldehyde was isolated as a white solid (55%, 118.8 g). ¹H NMR (250 MHz/CDCl₃); 2.32 (d, 6H, 2×CH₃, J=5.0 Hz), 9.33 (q, 2H, 2×CH, J=2.5 Hz). ¹³C NMR (60 MHz/CDCl₃); 51.1 (2×CH₃), 199.4 (2×C═O).

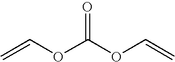

A mixture of mercuric diacetaldehyde (110 g, 381.02 mmol) in anhydrous THF (40 mL) was placed in a 250 mL three-necked flask equipped with a magnetic stirrer, thermometer, reflux condenser fitted with a CaCl₂ guard tube and an addition funnel. The mixture was stirred and cooled to 0° C. in an ice bath. Phosgene (14 g, 140 mmol) in anhydrous toluene (20%) was added gradually over a period of 20 minutes. The temperature was maintained at 0° C. for 1 hour with good stirring and then allowed to warm to room temperature for a further 1 hour. After this time the temperature was slowly raised to 60° C. and maintained for 1 hour. The product, divinyl carbonate, was obtained as a solution in toluene by distillation at atmospheric pressure. 1H NMR (250 MHz/CDCl₃); 4.95 (dd, 2H, 2×CH═CH trans, J=6.0, 2.0 Hz), 5.35 (dd, 2H, 2×CH═CH cis, J=14.0, 2.0 Hz), 7.50 (dd, 2H, 2×CH═CH₂, J=14.0, 6.0 Hz). ¹³C NMR (60 MHz/CDCl₃); 98.5 (CH═CH₂), 99.2 (CH═CH₂), 143.1 (2×CH═CH₂), 151.1 (C═O).

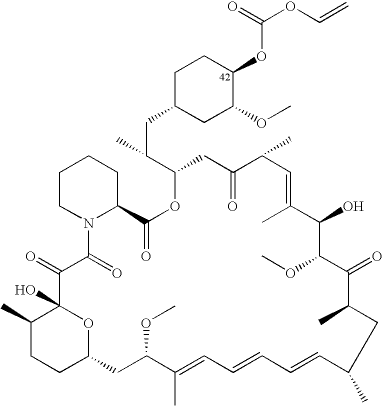

A mixture of rapamycin (0.03 g, 0.0328 mmol), divinyl carbonate (0.022 g, 0.197 mmol) and Novozyme 435 (0.045 g) in anhydrous tert-butyl methyl ether (TBME) (0.5 mL) and anhydrous toluene (0.13 mL) was stirred at 60° C. under an N₂ atmosphere for 18 hours. The enzyme was filtered off, washed with TBME and the combined organic solvent concentrated under N₂. The residue was purified by column chromatography (hexane/acetone, 3:1) to furnish the title compound as a white solid (88%, 0.0283 g). R_{f}=0.62 (THF/heptane, 1:1). ¹H NMR (400 MHz/CDCl₃); 4.54-4.58 (m, 1H), 4.58 (dd, 1H), 4.92 (dd, 1H), 7.11 (dd, 1H). ¹³C NMR (100 MHz/CDCl₃); 80.9, 97.8, 142.9, 152.6. MS (ESI-TOF) m/z 1006.6 [M+Na]⁺.

### Example 10

### 42-O-(Allyloxycarbonyl)rapamycin

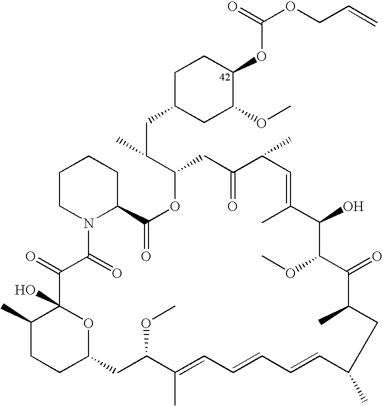

A mixture of rapamycin (0.03 g, 0.0328 mmol), diallyl carbonate (0.028 g, 0.197 mmol), Novozyme 435 (0.045 g) and molecular sieves (5 Å) in anhydrous tert-butyl methyl ether (TBME) (0.5 mL) was stirred at 60° C. under an N₂ atmosphere for 18 hours. The enzyme was filtered off, washed with TBME and the combined organic solvent concentrated under N₂. The residue was purified by column chromatography (hexane/acetone, 2.5:1) to furnish the title compound as a white solid (73%, 0.024 g). R_{f}=0.58 (THF/heptane, 1:1). ¹H NMR (400 MHz/CDCl₃); 4.50-4.57 (m, 1H), 4.63 (d, 2H), 5.27 (dd, 2H), 5.91-5.98 (m, 1H). ¹³C NMR (100 MHz/CDCl₃); 68.3, 80.3, 118.8, 131.7, 154.6. MS (ESI-TOF) m/z 1020.5 [M+Na]⁺.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. All patents, patent applications, and publications referenced herein are hereby incorporated by reference herein.

What is claimed is:

1. A compound comprising structure (I), (II), or (III) 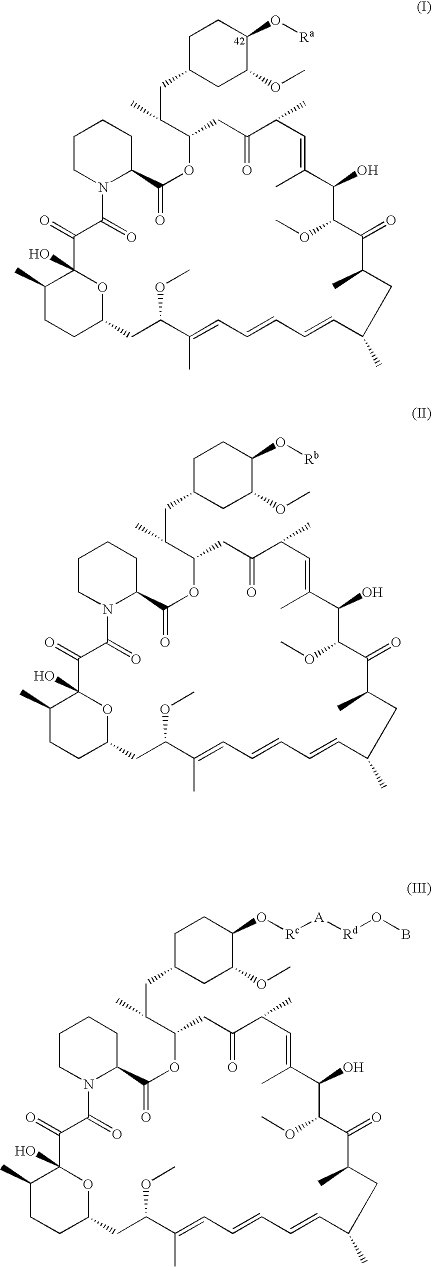
wherein:
R^{a} is —C(═O)OR¹, —C(═S)OR¹, —C(═O)SR¹, or —C(═S)SR¹ and,
R¹ is alkyl, alkenyl, alkynyl, cycloalkyl, heteroalkyl, heteroalkenyl, heteroalkynyl, heterocyclyl, or Ar—(CH₂)ₙ— where n is 0 to 12 and Ar is phenyl, substituted phenyl, aryl, heteroaryl, or substituted aryl, where at least one hydrogen of R¹ is replaced by a hydroxyl group, an amino group, a thio group, a phosphate group, a carbonyl group, a sulfonate group, a sulfonamide group, a sulfamide group, a carbonate group or a combination thereof,
R^{b} is —C(═O)OR², —C(═S)OR², —C(═O)SR², or —C(═S)SR² and,
R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group,
wherein the aromatic group is naphthyl, biphenyl, anthryl, tetrahydronaphthyl, phenanthryl, indane, tetracenyl, pentacenyl, triphenylenyl, imidazolyl, triazolyl, tetrazolyl, oxazolyl, isoxazolyl, thiazolyl, isothiazolyl, thiofuranyl, pyrimidinyl, pyrazinyl, thiazinyl, phenyl substituted with amine, phosphoryl, phosphate, sulfonyl, sulfonate, sulfonamide,
R^{c} and R^{d} are each independently —C(═O)O—, —C(═S)O—, —C(═O)S—, or —C(═S)S—,
A is alkyl, alkenyl, alkynyl, cycloalkyl, heteroalkyl, heteroalkenyl, heteroalkynyl, heterocyclyl, and —(CH₂)ₙ—Ar—(CH₂)ₙ— where n is 0 to 12 and Ar is phenyl, substituted phenyl, aryl, heteroaryl, or substituted aryl and,
B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl,
wherein alkyl is a linear or branched saturated aliphatic hydrocarbon group, alkenyl is a linear, branched or cyclic alkyl group containing at least one carbon-carbon double bond, alkynyl is a linear, branched or cyclic alkyl group containing at least one carbon-carbon triple bond, cycloalkyl is a cyclic saturated aliphatic hydrocarbon group, heteroalkyl is a linear or branched saturated aliphatic hydrocarbon group with at least one carbon atom being replaced by an atom other than carbon, heteroalkenyl is a linear, branched or cyclic alkyl group containing at least one carbon-carbon double bond with at least one carbon atom being replaced by an atom other than carbon, heteroalkynyl is a linear, branched or cyclic alkyl group containing at least one carbon-carbon triple bond with at least one carbon atom being replaced by an atom other than carbon, heterocyclyl is a cyclic saturated aliphatic hydrocarbon group with at least one carbon atom being replaced by an atom other than carbon,
or a pharmaceutically acceptable salt thereof.

2. The compound comprising claim 1, wherein at least one hydrogen of R¹ is replaced by a hydroxyl group.

3. A process of making a compound comprising structure (IX), 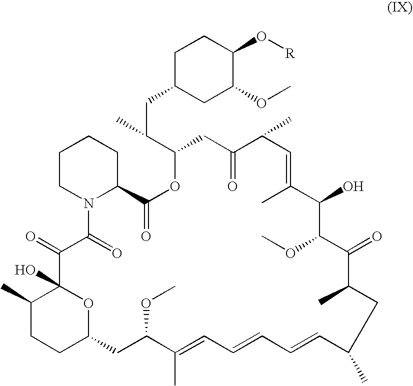
comprising the steps of:
(1) obtain a donor, and
(2) react the donor with an unprotected hydroxyl group at the '42 position of rapamycin or derivative thereof in the presence of a lipase to make an adduct.
wherein:
R is —C(═O)OR′, —C(═S)OR—, —C(═O)SR—, or —C(═S)SR— and,
R^{′} is alkyl, alkenyl, alkynyl, cycloalkyl, heteroalkyl, heteroalkenyl, heteroalkynyl, heterocyclyl, or Ar—(CH₂)ₙ— where n is 0 to 12 and Ar is phenyl, substituted phenyl, aryl, heteroaryl, or substituted aryl.

4. The process of claim 3 wherein the donor is a carbonate, O,O′-thiocarbonate, O,S-thiocarbonate, O,S-dithiocarbonate, or S,S′-dithiocarbonate donor of a general structure (IV) 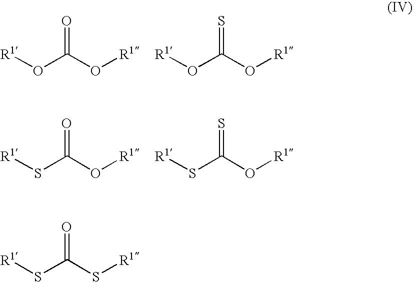
wherein:
R^{1′} is alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl, where at least one hydrogen of R^{1′} is replaced by a hydroxyl group, an amino group, a thio group, a phosphate group, a carbonyl group, a sulfonate group, a sulfonamide group, a sulfamide group, a carbonate group or a combination thereof,
wherein alkyl is a linear or branched saturated aliphatic hydrocarbon group, alkenyl is a linear, branched or cyclic alkyl group containing at least one carbon-carbon double bond, alkynyl is a linear, branched or cyclic alkyl group containing at least one carbon-carbon triple bond, cycloalkyl is a cyclic saturated aliphatic hydrocarbon group, heteroalkyl is a linear or branched saturated aliphatic hydrocarbon group with at least one carbon atom being replaced by an atom other than carbon, heteroalkenyl is a linear, branched or cyclic alkyl group containing at least one carbon-carbon double bond with at least one carbon atom being replaced by an atom other than carbon, heteroalkynyl is a linear, branched or cyclic alkyl group containing at least one carbon-carbon triple bond with at least one carbon atom being replaced by an atom other than carbon, heterocyclyl is a cyclic saturated aliphatic hydrocarbon group with at least one carbon atom being replaced by an atom other than carbon, and
the hydroxyl group, the amino group, the thio group, the phosphate group, the carbonyl group, the sulfonate group, the sulfonamide group, the sulfamide group, or the carbonate group is protected by a protection group, and
R^{1″} is a vinyl, ethyl, propyl, butyl, pentyl, hexyl, heptyl, octyl, or allyl group.

5. The process of claim 4 wherein the process further comprising the step of removing the protection group from the hydroxyl group, the amino group, the thio group, the phosphate group, the carbonyl group, the sulfonate group, the sulfonamide group, the sulfamide group, the carbonate group or a combination thereof to make a derivative of rapamycin comprising structure (I) from the adduct, 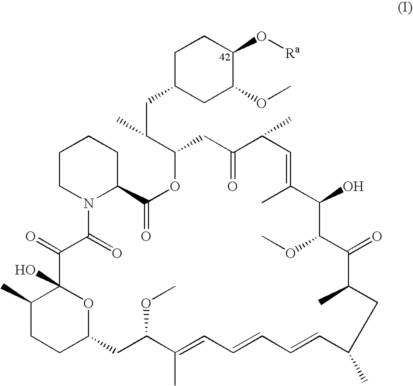
wherein,
R^{a} is —C(═O)OR¹, —C(═S)OR′, —C(═O)SR¹, or —C(═S)SR¹ and,
R¹ is alkyl, alkenyl, alkynyl, cycloalkyl, heteroalkyl, heteroalkenyl, heteroalkynyl, heterocyclyl, or Ar—(CH₂)ₙ— where n is 0 to 12 and Ar is phenyl, substituted phenyl, aryl, heteroaryl, or substituted aryl, where at least one hydrogen of R¹ is replaced by a hydroxyl group, an amino group, a thio group, a phosphate group, a carbonyl group, a sulfonate group, a sulfonamide group, a sulfamide group, a carbonate group or a combination thereof.

6. The process of claim 4 wherein at least one hydrogen of R^{1′} is replaced by a hydroxyl group.

7. The process of claim 4 wherein R^{1″} is a vinyl group.

8. The process of claim 3 wherein the donor is a carbonate, O,O′-thiocarbonate, O,S-thiocarbonate, O,S-dithiocarbonate, or S,S′-dithiocarbonate donor of a general structure (V) 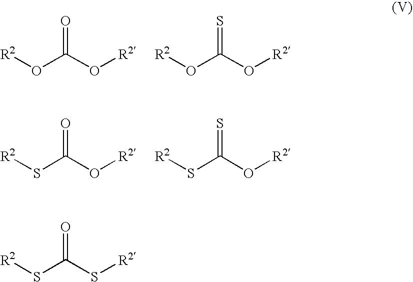
wherein:
R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group,
wherein the aromatic group is Ar—(CH₂)ₙ— where n is 0 to 12 and Ar is phenyl, substituted phenyl, aryl, heteroaryl, or substituted aryl, naphthyl, biphenyl, anthryl, tetrahydronaphthyl, phenanthryl, indane, tetracenyl, pentacenyl, triphenylenyl, imidazolyl, triazolyl, tetrazolyl, oxazolyl, isoxazolyl, thiazolyl, isothiazolyl, thiofuranyl, pyrimidinyl, pyrazinyl, thiazinyl, phenyl substituted with amine, phosphoryl, phosphate, sulfonyl, sulfonate, sulfonamide,
wherein alkyl is a linear or branched saturated aliphatic hydrocarbon group, alkenyl is a linear, branched or cyclic alkyl group containing at least one carbon-carbon double bond, alkynyl is a linear, branched or cyclic alkyl group containing at least one carbon-carbon triple bond, cycloalkyl is a cyclic saturated aliphatic hydrocarbon group, heteroalkyl is a linear or branched saturated aliphatic hydrocarbon group with at least one carbon atom being replaced by an atom other than carbon, heteroalkenyl is a linear, branched or cyclic alkyl group containing at least one carbon-carbon double bond with at least one carbon atom being replaced by an atom other than carbon, heteroalkynyl is a linear, branched or cyclic alkyl group containing at least one carbon-carbon triple bond with at least one carbon atom being replaced by an atom other than carbon, heterocyclyl is a cyclic saturated aliphatic hydrocarbon group with at least one carbon atom being replaced by an atom other than carbon, and
R^{2″} is a vinyl, ethyl, propyl, butyl, pentyl, hexyl, heptyl, octyl, or allyl group.

9. The process of claim 8 wherein R^{2′} is a vinyl group.

10. The process of claim 8 wherein the adduct comprising structure (II), 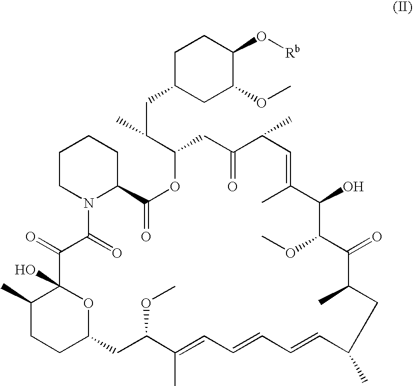
wherein,
R^{b} is —C(═O)OR², —C(═S)OR², —C(═O)SR², or —C(═S)SR² and,
R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group.

11. The process of claim 3 wherein the donor is symmetric, asymmetric or cyclic carbonates including alkyl carbonates, dialkyl carbonates, vinyl carbonates, divinyl carbonates, alkyl vinyl carbonates, and cyclic carbonates.

12. The process of claim 11 wherein the donor is diethyl carbonate, dioctyl carbonate, ethyl octyl carbonate, diallyl carbonate, cis-octadec-9-enyl vinyl carbonate, divinyl carbonate, 1,4-bis(vinylcarbonate)butane, 1,3-dioxan-2-one, 3-(trimethylsilyloxy)propyl vinyl carbonate, 3-(tert-butyldimethylsilyloxy)propyl vinyl carbonate, (S)-2,2-dimethyl-1,3-dioxolan-4-methyl vinyl carbonate and (S)-2,2-dimethyl-1,3-dioxolan-4-methyl ethyl carbonate.

13. The process of claim 3 wherein the donor is a bifunctional donor of a general structure (VI), 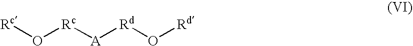
wherein:
R^{c} and R^{d} are each independently —C(═O)O—, —C(═S)O—, —C(═O)S—, or —C(═S)S—,
A is alkyl, alkenyl, alkynyl, cycloalkyl, heteroalkyl, heteroalkenyl, heteroalkynyl, heterocyclyl, and —(CH₂)ₙ—Ar—(CH₂)ₙ— where n is 0 to 12 and Ar is phenyl, substituted phenyl, aryl, heteroaryl, or substituted aryl,
wherein alkyl is a linear or branched saturated aliphatic hydrocarbon group, alkenyl is a linear, branched or cyclic alkyl group containing at least one carbon-carbon double bond, alkynyl is a linear, branched or cyclic alkyl group containing at least one carbon-carbon triple bond, cycloalkyl is a cyclic saturated aliphatic hydrocarbon group, heteroalkyl is a linear or branched saturated aliphatic hydrocarbon group with at least one carbon atom being replaced by an atom other than carbon, heteroalkenyl is a linear, branched or cyclic alkyl group containing at least one carbon-carbon double bond with at least one carbon atom being replaced by an atom other than carbon, heteroalkynyl is a linear, branched or cyclic alkyl group containing at least one carbon-carbon triple bond with at least one carbon atom being replaced by an atom other than carbon, heterocyclyl is a cyclic saturated aliphatic hydrocarbon group with at least one carbon atom being replaced by an atom other than carbon, and
R^{c′} and R^{d′} are each independently a vinyl, ethyl, propyl, butyl, pentyl, hexyl, heptyl, octyl, or allyl group.

14. The process of claim 13 wherein R_{c′} and R^{d′} are each independently a vinyl group.

15. The process of claim 13 wherein the bifunctional donor is 1,4-bis(vinylcarbonate)butane, 1,3-bis(vinylcarbonate)propane, 1,2-bis(vinylcarbonate)ethane, 1,4-bis(ethylcarbonate)butane, 1,3-bis(ethylcarbonate)propane, 1,2-bis(ethylcarbonate)ethane, 1,4-bis(methyl vinyl carbonate)cyclohexane or 2,5-bis(methyl vinyl carbonate)furan.

16. The process of claim 13 wherein the process further comprise the step of reacting the adduct with a compound of formula B—OH, where OH is a hydroxyl group and B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl, to make a rapamycin derivative comprising structure (III).

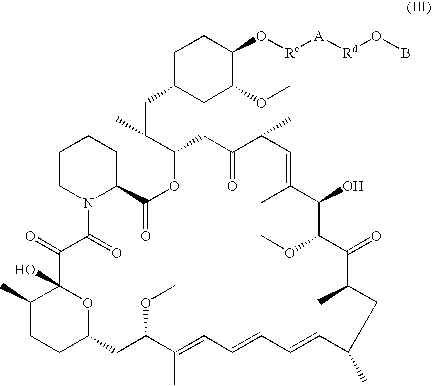

17. The process of claim 16 wherein the hydroxyl group in B—OH is the 42-hydroxyl of rapamycin or derivative thereof.

18. The process of claim 16 wherein B—OH is alkyl alcohols, alkenyl alcohols, alkynyl alcohols, aryl alcohols, diols, triols, polyols, cyclic alcohols, threitol, inositol, or polyethers.

19. The process of claim 3 wherein the lipase is Aspergillus niger lipase, Candida antarctica “A” lipase, Candida antarctica “B” lipase, Amano Lipase PS-C II, Candida rugosa lipase, Mucor miehei lipase, Pseudomonas cepacia lipase (lipase PS), Rhizopus delemar lipase, or alike.

20. The process of claim 3 wherein the lipase is Candida antarctica “B” lipase.

21. The process of claim 3 wherein the lipase is Amano Lipase PS-C II.

22. A method of treating a disease comprising administration to a subject in need thereof a therapeutically effective amount of a compound comprising the structure (I), (II), or (III) or a pharmaceutically acceptable salt thereof,
wherein:
the disease is organ and tissue transplant rejection, autoimmune disease, proliferative disorder, restenosis, cancer, or microbial infection,
R^{a} is —C(═O)OR¹, —C(═S)OR¹, —C(═O)SR¹, or —C(═S)SR¹,
R¹ is alkyl, alkenyl, alkynyl, cycloalkyl, heteroalkyl, heteroalkenyl, heteroalkynyl, heterocyclyl, or Ar—(CH₂)ₙ— where n is 0 to 12 and Ar is phenyl, substituted phenyl, aryl, heteroaryl, or substituted aryl, where at least one hydrogen of R¹ is replaced by a hydroxyl group, an amino group, a thio group, a phosphate group, a carbonyl group, a sulfonate group, a sulfonamide group, a sulfamide group, a carbonate group or a combination thereof,
R^{b} is —C(═O)OR², —(C═S)OR², —C(═O)SR², or —C(═S)SR²,
R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group,
wherein the aromatic group is naphthyl, biphenyl, anthryl, tetrahydronaphthyl, phenanthryl, indane, tetracenyl, pentacenyl, triphenylenyl, imidazolyl, triazolyl, tetrazolyl, oxazolyl, isoxazolyl, thiazolyl, isothiazolyl, thiofuranyl, pyrimidinyl, pyrazinyl, thiazinyl, phenyl substituted with amine, phosphoryl, phosphate, sulfonyl, sulfonate, sulfonamide,
R^{c} and R^{d} are each independently —C(═O)O—, —C(═S)O—, —C(═O)S—, or —C(═S)S—,
A is alkyl, alkenyl, alkynyl, cycloalkyl, heteroalkyl, heteroalkenyl, heteroalkynyl, heterocyclyl, and —(CH₂)ₙ—Ar—(CH₂)ₙ— where n is 0 to 12 and Ar is phenyl, substituted phenyl, aryl, heteroaryl, or substituted aryl and,
B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl,
wherein alkyl is a linear or branched saturated aliphatic hydrocarbon group, alkenyl is a linear, branched or cyclic alkyl group containing at least one carbon-carbon double bond, alkynyl is a linear, branched or cyclic alkyl group containing at least one carbon-carbon triple bond, cycloalkyl is a cyclic saturated aliphatic hydrocarbon group, heteroalkyl is a linear or branched saturated aliphatic hydrocarbon group with at least one carbon atom being replaced by an atom other than carbon, heteroalkenyl is a linear, branched or cyclic alkyl group containing at least one carbon-carbon double bond with at least one carbon atom being replaced by an atom other than carbon, heteroalkynyl is a linear, branched or cyclic alkyl group containing at least one carbon-carbon triple bond with at least one carbon atom being replaced by an atom other than carbon, heterocyclyl is a cyclic saturated aliphatic hydrocarbon group with at least one carbon atom being replaced by an atom other than carbon.

23. A pharmaceutical composition for treating a disease comprising a pharmaceutical carrier and an effective amount of a compound comprising the structure (I), (II), or (III) or a pharmaceutically acceptable salt thereof,
wherein:
the disease is organ and tissue transplant rejection, autoimmune disease, proliferative disorder, restenosis, cancer, or microbial infection,
R^{a} is —C(═O)OR¹, —C(═S)OR¹, —C(═O)SR¹, or —C(═S)SR¹,
R¹ is alkyl, alkenyl, alkynyl, cycloalkyl, heteroalkyl, heteroalkenyl, heteroalkynyl, heterocyclyl, or Ar—(CH₂)ₙ— where n is 0 to 12 and Ar is phenyl, substituted phenyl, aryl, heteroaryl, or substituted aryl, where at least one hydrogen of R¹ is replaced by a hydroxyl group, an amino group, a thio group, a phosphate group, a carbonyl group, a carbonate group, a sulfonate group, a sulfonamide group, a sulfamide group, or a combination thereof,
R^{b} is —C(═O)OR², —C(═S)OR², —C(═O)SR², or —C(═S)SR²,
R² is C₇-C₂₂ alkyl, C₇-C₂₂ alkenyl, C₂-C₂₂ alkynyl, C₉-C₁₂ cycloalkyl, heteroalkyl, heteroalkenyl, heteroalkynyl, heterocyclyl, or an aromatic group,
wherein the aromatic group is naphthyl, biphenyl, anthryl, tetrahydronaphthyl, phenanthryl, indane, tetracenyl, pentacenyl, triphenylenyl, imidazolyl, triazolyl, tetrazolyl, oxazolyl, isoxazolyl, thiazolyl, isothiazolyl, thiofuranyl, pyrimidinyl, pyrazinyl, thiazinyl, phenyl substituted with amine, phosphoryl, phosphate, sulfonyl, sulfonate, sulfonamide,
R^{c} and R^{d} are each independently —C(═O)O—, —C(═S)O—, —C(═O)S—, or —C(═S)S—,
A is alkyl, alkenyl, alkynyl, cycloalkyl, heteroalkyl, heteroalkenyl, heteroalkynyl, heterocyclyl, and —(CH₂)ₙ—Ar—(CH₂)ₙ— where n is 0 to 12 and Ar is phenyl, substituted phenyl, aryl, heteroaryl, or substituted aryl and,
B is rapamycin or derivative thereof, alkyl, alkenyl, alkynyl, cycloalkyl, heteroalkyl, heteroalkenyl, heteroalkynyl, heterocyclyl, and Ar—(CH₂)ₙ— where n is 0 to 12 and Ar is phenyl, substituted phenyl, aryl, heteroaryl, or substituted aryl,
wherein alkyl is a linear or branched saturated aliphatic hydrocarbon group, alkenyl is a linear, branched or cyclic alkyl group containing at least one carbon-carbon double bond, alkynyl is a linear, branched or cyclic alkyl group containing at least one carbon-carbon triple bond, cycloalkyl is a cyclic saturated aliphatic hydrocarbon group, heteroalkyl is a linear or branched saturated aliphatic hydrocarbon group with at least one carbon atom being replaced by an atom other than carbon, heteroalkenyl is a linear, branched or cyclic alkyl group containing at least one carbon-carbon double bond with at least one carbon atom being replaced by an atom other than carbon, heteroalkynyl is a linear, branched or cyclic alkyl group containing at least one carbon-carbon triple bond with at least one carbon atom being replaced by an atom other than carbon, heterocyclyl is a cyclic saturated aliphatic hydrocarbon group with at least one carbon atom being replaced by an atom other than carbon.

24. The compound of claim 1 wherein the compound is 42-O-[(4′-vinyl carbonate)but-1′-oxycarbonyl]rapamycin, 42-O-(cis-octadec-9′-enyloxycarbonyl)rapamycin, 42-O—[(S)-2′,3′-dihydroxypropyloxycarbonyl]rapamycin, 42-O-(3′-hydroxypropoxycarbonyl) rapamycin, 42-O-[(4′-dodecyl carbonate)but-1′-oxycarbonyl]rapamycin.

25. The process of claim 3 wherein the compound is 42-O-[(4′-vinyl carbonate)but-1′-oxycarbonyl]rapamycin, 42-O-(cis-octadec-9′-enyloxycarbonyl)rapamycin, 42-O—[(S)-2′,3′-dihydroxypropyloxycarbonyl]rapamycin, 42-O-(3′-hydroxypropoxycarbonyl)rapamycin, 42-O-[(4′-dodecyl carbonate)but-1′-oxycarbonyl]rapamycin.

26. The method of claim 22, wherein the compound is 42-O-[(4′-vinyl carbonate)but-1′-oxycarbonyl]rapamycin, 42-O-(cis-octadec-9′-enyloxycarbonyl)rapamycin, 42-O—[(S)-2′,3′-dihydroxypropyloxycarbonyl]rapamycin, 42-O-(3′-hydroxypropoxycarbonyl)rapamycin, 42-O-[(4′-dodecyl carbonate)but-1′-oxycarbonyl]rapamycin.

27. The pharmaceutical composition of claim 23, wherein the compound is 42-O-[(4′-vinyl carbonate)but-1′-oxycarbonyl]rapamycin, 42-O-(cis-octadec-9′-enyloxycarbonyl)rapamycin, 42-O—[(S)-2′,3′-dihydroxypropyloxycarbonyl]rapamycin, 42-O-(3′-hydroxypropoxycarbonyl)rapamycin, 42-O-[(4′-dodecyl carbonate)but-1′-oxycarbonyl]rapamycin.
